SEQUENCE LISTING (6 sequences)

SEQ ID NO: 1 - DNA, 691 bp, Homo Sapiens
gacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaac

SEQ ID NO: 2 - protein, 175 aa, Homo Sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 3 - DNA, 865 bp, Homo Sapiens
ctcaaacaccgcctgctaaaaatacccgactggaggagcataaaagcgcagccgagcccagcgccccgcacttttctgagcagacgtccagagcagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaaagccttagcctggatgcccacccctgctgccgccactggctgtgcctcccccgccacctgtgtgttcttttgatacatttatcttctgtttttctcaaataaagttcaaagcaaccacctgtaaaaaaaaaaaaaaaaaa

SEQ ID NO: 4 - protein, 205 aa, Homo Sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 5 - DNA, 5087 bp, Homo Sapiens
aacgggcgccgcggcggggagaagacgcagagcgctgctgggctgccgggtctcccgcttccccctcctgctccaagggcctcctgcatgagggcgcggtagagacccggacccgcgccgtgctcctgccgtttcgctgcgctccgcccgggcccggctcagccaggccccgcggtgagccatgattcgcctcggggctccccagacgctggtgctgctgacgctgctcgtcgccgctgtccttcggtgtcagggccaggatgtccaggaggctggcagctgtgtgcaggatgggcagaggtataatgataaggatgtgtggaagccggagccctgccggatctgtgtctgtgacactgggactgtcctctgcgacgacataatctgtgaagacgtgaaagactgcctcagccctgagatccccttcggagagtgctgccccatctgcccaactgacctcgccactgccagtgggcaaccaggaccaaagggacagaaaggagaacctggagacatcaaggatattgtaggacccaaaggacctcctgggcctcagggacctgcaggggaacaaggacccagaggggatcgtggtgacaaaggtgaaaaaggtgcccctggacctcgtggcagagatggagaacctgggacccctggaaatcctggcccccctggtcctcccggcccccctggtccccctggtcttggtggaaactttgctgcccagatggctggaggatttgatgaaaaggctggtggcgcccagttgggagtaatgcaaggaccaatgggccccatgggacctcgaggacctccaggccctgcaggtgctcctgggcctcaaggatttcaaggcaatcctggtgaacctggtgaacctggtgtctctggtcccatgggtccccgtggtcctcctggtccccctggaaagcctggtgatgatggtgaagctggaaaacctggaaaagctggtgaaaggggtccgcctggtcctcagggtgctcgtggtttcccaggaaccccaggccttcctggtgtcaaaggtcacagaggttatccaggcctggacggtgctaagggagaggcgggtgctcctggtgtgaagggtgagagtggttccccgggtgagaacggatctccgggcccaatgggtcctcgtggcctgcctggtgaaagaggacggactggccctgctggcgctgcgggtgcccgaggcaacgatggtcagccaggccccgcagggcctccgggtcctgtcggtcctgctggtggtcctggcttccctggtgctcctggagccaagggtgaagccggccccactggtgcccgtggtcctgaaggtgctcaaggtcctcgcggtgaacctggtactcctgggtcccctgggcctgctggtgcctccggtaaccctggaacagatggaattcctggagccaaaggatctgctggtgctcctggcattgctggtgctcctggcttccctgggccacggggccctcctggccctcaaggtgcaactggtcctctgggcccgaaaggtcagacgggtgaacctggtattgctggcttcaaaggtgaacaaggccccaagggagaacctggccctgctggcccccagggagcccctggacccgctggtgaagaaggcaagagaggtgcccgtggagagcctggtggcgttgggcccatcggtccccctggagaaagaggtgctcccggcaaccgcggtttcccaggtcaagatggtctggcaggtcccaagggagcccctggagagcgagggcccagtggtcttgctggccccaagggagccaacggtgaccctggccgtcctggagaacctggccttcctggagcccggggtctcactggccgccctggtgatgctggtcctcaaggcaaagttggcccttctggagcccctggtgaagatggtcgtcctggacctccaggtcctcagggggctcgtgggcagcctggtgtcatgggtttccctggccccaaaggtgccaacggtgagcctggcaaagctggtgagaagggactgcctggtgctcctggtctgaggggtcttcctggcaaagatggtgagacaggtgctgcaggaccccctggccctgctggacctgctggtgaacgaggcgagcagggtgctcctgggccatctgggttccagggacttcctggccctcctggtcccccaggtgaaggtggaaaaccaggtgaccagggtgttcccggtgaagctggagcccctggcctcgtgggtcccaggggtgaacgaggtttcccaggtgaacgtggctctcccggtgcccagggcctccagggtccccgtggcctccccggcactcctggcactgatggtcccaaaggtgcatctggcccagcaggcccccctggggctcagggccctccaggtcttcagggaatgcctggcgagaggggagcagctggtatcgctgggcccaaaggcgacaggggtgacgttggtgagaaaggccctgagggagcccctggaaaggatggtggacgaggcctgacaggtcccattggcccccctggcccagctggtgctaatggcgagaagggagaagttggacctcctggtcctgcaggaagtgctggtgctcgtggcgctccgggtgaacgtggagagactgggccccccggaccagcgggatttgctgggcctcctggtgctgatggccagcctggggccaagggtgagcaaggagaggccggccagaaaggcgatgctggtgcccctggtcctcagggcccctctggagcacctgggcctcagggtcctactggagtgactggtcctaaaggagcccgaggtgcccaaggccccccgggagccactggattccctggagctgctggccgcgttggacccccaggctccaatggcaaccctggaccccctggtccccctggtccttctggaaaagatggtcccaaaggtgctcgaggagacagcggcccccctggccgagctggtgaacccggcctccaaggtcctgctggaccccctggcgagaagggagagcctggagatgacggtccctctggtgccgaaggtccaccaggtccccagggtctggctggtcagagaggcatcgtcggtctgcctgggcaacgtggtgagagaggattccctggcttgcctggcccgtcgggtgagcccggcaagcagggtgctcctggagcatctggagacagaggtcctcctggccccgtgggtcctcctggcctgacgggtcctgcaggtgaacctggacgagagggaagccccggtgctgatggcccccctggcagagatggcgctgctggagtcaagggtgatcgtggtgagactggtgctgtgggagctcctggagcccctgggccccctggctcccctggccccgctggtccaactggcaagcaaggagacagaggagaagctggtgcacaaggccccatgggaccctcaggaccagctggagcccggggaatccagggtcctcaaggccccagaggtgacaaaggagaggctggagagcctggcgagagaggcctgaagggacaccgtggcttcactggtctgcagggtctgcccggccctcctggtccttctggagaccaaggtgcttctggtcctgctggtccttctggccctagaggtcctcctggccccgtcggtccctctggcaaagatggtgctaatggaatccctggccccattgggcctcctggtccccgtggacgatcaggcgaaaccggccctgctggtcctcctggaaatcctggaccccctggtcctccaggtccccctggccctggcatcgacatgtccgcctttgctggcttaggcccgagagagaagggccccgaccccctgcagtacatgcgggccgaccaggcagccggtggcctgagacagcatgacgccgaggtggatgccacactcaagtccctcaacaaccagattgagagcatccgcagccccgagggctcccgcaagaaccctgctcgcacctgcagagacctgaaactctgccaccctgagtggaagagtggagactactggattgaccccaaccaaggctgcaccttggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacgaaacataccggtaagtggggcaagactgttatcgagtaccggtcacagaagacctcacgcctccccatcattgacattgcacccatggacataggagggcccgagcaggaattcggtgtggacatagggccggtctgcttcttgtaaaaacctgaacccagaaacaacacaatccgttgcaaacccaaaggacccaagtactttccaatctcagtcactctaggactctgcactgaatggctgacctgacctgatgtccattcatcccaccctctcacagttcggacttttctcccctctctttctaagagacctgaactgggcagactgcaaaataaaatctcggtgttctatttatttattgtcttcctgtaagaccttcgggtcaaggcagaggcaggaaactaactggtgtgagtcaaatgccccctgagtgactgcccccagcccaggccagaagacctcccttcaggtgccgggcgcaggaactgtgtgtgtcctacacaatggtgctattctgtgtcaaacacctctgtattttttaaaacatcaattgatattaaaaatgaaaagattattggaaagtaca

SEQ ID NO: 6 - protein, 1487 aa, Homo Sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

(19) United States
(12) Patent Application Publication
(10) Pub. No.: US 2010/0330036 A1
(43) Pub. Date: Dec. 30, 2010

(54) MARKERS OF MATRIX GENE EXPRESSION AND CELLULAR DIFFERENTIATION IN CHONDROCYTES

(75) Inventors: Stijn Lambrecht, Melle (BE); Dieter Deforce, Kuurne (BE); Dirk Elewaut, Heusden (BE); August Verbruggen, Gent (BE)

Correspondence Address: DINSMORE & SHOHL LLP, FIFTH THIRD CENTER, ONE SOUTH MAIN STREET, SUITE 1300, DAYTON, OH, 45402-2023, US

(73) Assignee: UNIVERSITEIT GENT, Gent (BE)

(21) Appl. No.: 12/738,398

(22) PCT Filed: Oct. 20, 2008
(86) PCT No.: PCT/EP08/08869

§ 371 Date: May 17, 2010

(30) Foreign Application Priority Data
Oct. 18, 2007 (GB) 0720502.4

(51) Int. Cl. A61K 35/12; C12Q 1/02; C12N 5/071; C12Q 1/68; A61P 19/02

(52) U.S. Cl. 424/93.1; 435/29; 435/325; 435/6

(57) ABSTRACT

The invention relates generally to the field of tissue engineering. More particularly, the present invention relates to methods for identifying a population of cells suitable for the repair of connective tissue, including cartilage. The invention further provides methods and compositions related to the generation of a population of cells suitable for the repair of cartilage, in particular in the repair of cartilage degeneration associated with osteoarthritis. Methods of using said cells thus identified or thus generated, in methods to extend the period of cell manipulation, i.e. to increase the yield of cells suitable in the aforementioned methods, are also provided by the present invention.

### FIELD OF THE INVENTION

The invention relates generally to the field of tissue engineering. More particularly, the present invention relates to methods for identifying a population of cells suitable for the repair of connective tissue, including cartilage.

The invention further provides methods and compositions related to the generation of a population of cells suitable for the repair of cartilage, in particular in the repair of cartilage degeneration associated with osteoarthritis.

Methods of using said cells thus identified or thus generated, in methods to extend the period of cell manipulation, i.e. to increase the yield of cells suitable in the aforementioned methods, are also provided by the present invention.

### BACKGROUND TO THE INVENTION

Osteoarthritis (OA) has become one of the major health problems in the western world [1]. The key hallmark of this disease is a slow progressive degeneration of the articular cartilage [2]. Articular cartilage forms a specialized, smooth connective tissue that is weight bearing and that serves as a gliding surface allowing a lithe movement of the joints. The majority of the cartilage tissue is formed by highly abundant extracellular-matrix (ECM) components [3]. Chondrocytes, the only resident cells in articular cartilage represents only 1% of the cartilage volume. It is however, generally accepted that these cells are the key players in cartilage degeneration associated with OA. The chondrocytes metabolism is influenced by a variety of cytokines and growth factors. Some are considered to be anabolic factors (IGF-I, TGF-b, BMPs), others are catabolic (IL-1, TNF-a). The balance between these anabolic and catabolic factors is of major importance to maintain a normal homeostasis in the chondrocytes metabolism. Until now, the knowledge of the processes involved in the pathogenesis of OA at the molecular level is limited. A better understanding of the molecular actors in the chondrocyte metabolism, may provide tools for the rational development of disease-modifying drugs.

Current treatments are based on the in vitro expansion and subsequent implantation of autologous chondrocytes obtained from an unaffected area of the joint. Where this procedure has led to a proven symptomatic amelioration, there is still a wide marging of improvement, in particular to the selection of and cell culture conditions for phenotypic stable cells.

It has been an object of the present invention to identify biomarkers/regulators of cartilage matrix gene expression and to provide improved methods to expand phenotypic stable cells suitable for the repair of connective tissue.

Here, we represent novel markers, in particular αBcrystallin (CRYAB), that may influence the chondrogenic capacity of the chondrocytes and that may serve as a marker for chondrogenic capacity and differentiation status of the chondrocyte. In contrast, to convential differentiation markers, which are typical end-point markers, the present marker is able to influence the expression of several of these end-point markers in differentiated chondrocytes (collagen type II (COL2A1), Bone Morphogenetic Protein 2 (BMP-2) and aggrecan), hereby suggesting an upstream location in biological pathways.

This early marker allows for monitoring chondrocyte-precursors, including chondrocyte derivatives, which but for CRYAB, express very low levels of or even no conventional chondrocyte differentiation markers such as BMP-2 and COL2A1, and is accordingly useful as alternative marker in methods to select phenotypic stable cells suitable for the repair of connective tissue.

### SUMMARY OF THE INVENTION

This invention is based on the identification of αBcrystallin (CRYAB) as molecular actor in the homeostasis of chondrocytes, and accordingly provides CRYAB as molecular marker useful in monitoring the phentotypic stability of isolated pluripotent skeletal cells in vitro.

In a first objective, the present invention provides a method to monitor and assess the phenotypic stability of a cell expansion culture of isolated pluripotent cells and/or chondrocytes, said method comprising determining the expression levels of CRYAB at regular time intervals.

It has in this respect been found that a transient expression of CRYAB marks a phenotypic change in cell expansion cultures of isolated pluripotent cells and/or chondrocytes. Differentiation of pluripotent cells into chondrocytes is marked with a transient increase of CRYAB expression, where dedifferentiation of isolated chondrocytes is accompanied with a transient decrease in CRYAB expression. The latter is particularly useful in monitoring the phenotypic stability a cell expansion culture of isolated chondrocytes, wherein the lowest CRYAB expression marks the start of a phenotypic change of said cells. As is shown in the examples hereinafter, the lowest CRYAB expression is accompanied with;
a decreased expression of positive markers of a chondrogenic phenotype, such as BMP-2 and matrix genes including collagen type II (COL2A1) and aggrecan; and an increased expression of negative markers of a chondrogenic phenotype, such as Activin receptor Like Kinase 1 (ALK-1).

Thus in one embodiment the present invention allows to monitor and identify phenotypic stable cell cultures, said method comprising monitoring the transient change in CRYAB expression, wherein the highest respectively lowest CRYAB expression within said transient change, marks the start of the phenotypic change of said cell culture.

In a particular embodiment the methods of the present invention allow to monitor and identify a phenotypic stable cell culture of isolated chondrocytes, said method comprising monitoring the transient decrease in CRYAB expression, wherein up to the increase of CRYAB expression, said cell culture is identified as a phenotypic stable cell culture.

As is evident from the examples hereinafter, and in one embodiment of the present invention, said transient decrease in CRYAB expression is at least 30%, 40% or 50% of the initial CRYAB expression; and in particular equals from about 35% to about 80% of the initial CRYAB expression; more in particular from about 40% to about 60% (i.e. to about 40%, 41%, 42%, 43%, 44%, 45%, 46%, 47%, 48%, 49%, 50%, 51%, 52%, 53%, 54%, 55%, 55%, 57%, 58%, 59%, 60%, 65% or 70% of the initial CRYAB expression) of the initial CRYAB expression in said cell culture of isolated pluripotent cells and/or chondrocytes.

Further to the characterization of CRYAB as a molecular actor in the homeostasis of chondrocytes, the present invention also identified HSP27 as a negative marker for the chondrogenic capacity of a culture of isolated chondrocytes.

Thus, in an alternative embodiment the method to monitor the phenotypic stability of a cell culture of isolated chondrocytes comprises determining HSP27 expression, wherein an increase in HSP27 expression is an indication of the dedifferentiation of said cell culture, i.e. of the phenotypic change of said cell culture.

In another alternative embodiment the method to monitor the phenotypic stability of a cell culture of isolated chondrocytes further comprises determining HSP27 and CRYAB expression, wherein an increase in HSP27 expression in combination with a transient decrease in CRYAB expression is an indication of the dedifferentiation of said cell culture, i.e. of the phenotypic change of said cell culture.

In said alternative embodiments an increase in HSP27 expression to a level of at least 140%; in particular up to about 150% to about 250% (i.e. up to about 150%, 155%, 160%, 165%, 170%, 175%, 180%, 185%, 190%, 195%, 200%, 205%, 210%, 215%, 220%, 225%, 230%, 235%, 240%, 245%, 250% of the initial HSP27 expression) of the initial HSP27 expression, is an indication of the point marking the dedifferentiation of said cell culture of isolated pluripotent cells and/or chondrocytes.

In a particular embodiment the cell culture of isolated pluripotent cells and/or chondrocytes is identified as a phenotypic stable cell culture;
up to a decrease in CRYAB expression down to about 70% or less (i.e. down to about 20%, 25%, 30%, 35%, 40%, 41%, 42%, 43%, 44%, 45%, 46%, 47%, 48%, 49%, 50%, 51%, 52%, 53%, 54%, 55%, 55%, 57%, 58%, 59%, 60%, 65% or 70% of the initial CRYAB expression) of the initial CRYAB expression; in particular down to about 60% to about 40% of the initial CRYAB expression; and
an increase in HSP27 expression up to about 140% or more of the initial HSP27 expression; in particular up to about 150% to about 250% (i.e. up to about 150%, 155%, 160%, 165%, 170%, 175%, 180%, 185%, 190%, 195%, 200%, 205%, 210%, 215%, 220%, 225%, 230%, 235%, 240%, 245%, 250% of the initial HSP27 expression) of the initial HSP27 expression

As is shown for example in FIG. 7, when the cell culture of isolated pluripotent cells and/or chondrocytes cells is kept beyond the lowest CRYAB expression within the transient change described herein, the cells lose the capability of expressing the typical markers of the chondrogenic phenotype like COL2A1 and aggrecan, upon reconstitution in their endogenous environment.

Thus, in an even further embodiment, the phenotypic stability of the cell culture of isolated pluripotent cells and/or chondrocytes, is further characterised in the capability of said cells to retain COL2A1 and aggrecan expression upon reconstitution in their endogenous environment.

As provided in more detail in the examples hereinafter, in one embodiment of the present invention the capability of said cells to express COL2A1 and/or aggrecan is tested at regular time intervals by 3D culturing of said cells on a suitable carrier using art known procedures such as described in “Cartilage repair: surgical techniques and tissue engineering using polysaccharide- and collagen-base d materials. Biorheology. 2004, 41 (3-4), 433-443. Suitable carriers include agarose, carboxymethyl cellulose and other cellulose derivatives, alginates, gelatin, and polyvinyl pyrolidone.

Alternatively, the ability of said cells to express COL2A1 expression is based on the change in COL2A1 expression in a cell expansion culture of said isolated pluripotent cells and/or chondrocytes. In addition to the above, i.e. the transient decrease in CRYAB expression and/or the increase in HSP27 expression, the point marking the dedifferentiation of said cell culture of isolated pluripotent cells and/or chondrocytes, is further characterized in a decrease of at least 30% of the initial COL2A1 expression; in particular a decrease with about 35% to about 70% (i.e. with about 35%, 40%, 45%, 50%, 55%, 60%, 65% or 70% of the initial COL2A1 expression) of the initial COL2A1 expression; more in particular with a decrease of about 40% to about 50% of the initial COL2A1 expression (i.e. with about 40%, 41%, 42%, 43%, 44% or 45% of the initial COL2A1 expression).

Based on the observations that the lowest CRYAB expression in the transient decrease is accompanied with a change in chondrogenic markers (and in particular with a change in COL2A1, and HSP27 expression) that suggest a decrease in chondrogenic capacity of said cell culture, it is a further object of the present invention to use the aforementioned markers in determining the chondrogenic capacity of a cell culture of isolated chondrocytes and to use the aforementioned methods in monitoring and identifying a cell culture of isolated chondrocytes with high chondrogenic capacity.

The methods as provided herein, are particularly useful in monitoring the phenotypic stability of a cell culture of isolated chondrocytes, i.e. chondrocytes derived from skeletal tissue, such as for example derived from hyaline cartilage or fibrocartilage, including hyaline knee cartilage chondrocytes, synovial fibroblasts and meniscal chondrocytes.

In a second objective, the present invention provides the use of the methods according to the invention, to identify cell culture conditions or treatments (compounds) that enhance the phenotypic stability of a cell culture. It is known for instance, that extensive cell expansion of isolated pluripotent skeletal cells, compromises the chondrogenic capacity of said cells. Some treatments, such as for example addition of growth factors/reagents, or cell culture conditions, such as for example non-adherent growth conditions, may influence the phenotypic stability of the cell culture. Monitoring the expression of CRYAB, optionally with HSP27, the present invention provides the tools to identify treatments and/or cell culture conditions that prevent or delay the dedifferentiation of the isolated pluripotent skeletal cells, and thus enhance the chondrogenic capacity of the in vitro culture of cells.

As a third embodiment the present invention provides methods to identify compounds capable of modulating the sensitivity of a cell culture of isolated pluripotent cells and/or chondrocytes for pro-inflamatory cytokines such as IL-1, IL-17, IL-18, IL-6, IL-8 or TNF, in particular IL-1 or TNF-α. These methods are based on the observation that CRYAB expression, HSP27 expression is reduced in a cell culture of isolated chondrocytes, upon treatment with pro-inflammatory cytokines. In one objective of this embodiment, the method to identify compounds capable of modulating the sensitivity of a cell culture of isolated chondrocytes comprises, determining the expression levels of CRYAB and/or HSP27 in a cell culture treated with a pro-inflammatory cytokine, both in the presence and absence of the compound to be tested, and determine whether the test compound has an effect on the expression level of CRYAB and/or HSP27 in said cell culture. A compound capable to change the expression levels of CRYAB and/or HSP27 in said cell culture is identified as a compound capable to modulate the pro-inflammatory sensitivity of said cells. In a particular embodiment, the method allows to identify compounds that reduce the sensitivity of a cell culture of isolated chondrocytes for pro-inflammatory cytokines, said method comprising determining the expression levels of CRYAB and/or HSP27 in a cell culture treated with a pro-inflammatory cytokine, both in the presence and absence of the compound to be tested, and determine whether the test compound increases the expression level of CRYAB and/or HSP27 in said cell culture.

As a fourth objective, the present invention provides the cell cultures with high chondrogenic capacity obtainable by any one of the methods of the present invention. Such cell cultures can be used in a variety of clinical applications including transplantation into a patient for the repair of connective tissue, in particular in the repair of cartilage, more in particular in the repair of cartilage degeneration associated with osteoarthritis. It is accordingly an object of the present invention to provide a pharmaceutical composition comprising cells obtained using the methods of the present invention.

It is also an objective of the present invention, to provide the use of the early chondrogenic marker CRYAB and of HSP27 as a negative marker for the chondrogenic capacity of a culture of isolated chondrocytes, in a method to assess the chondrogenic capacity of such a cell culture.

### BRIEF DESCRIPTION OF THE DRAWINGS

FIG. 1: A comparative 2-DE analysis (6 NoNo [chondrocytes isolated from cartilage from healthy donors], 7 NoOA [chondrocytes isolated from visually intact zones of OA-cartilage] and 7 OAOA samples [chondrocytes isolated from visually damaged zones of OA-cartilage]) showed the upregulation of a spot, identified as αBcrystallin, in healthy chondrocytes. Upper panel shows a zoomed image of two representative gels (left: NoNo-sample, right: OAOA sample). Lower panel: Bars represent the mean intensity of the spot (±SEM) in each group (relative to NoNo expression levels). *: Denotes statistical significance (p<0.05; Mann-Whitney U-test) compared to NoNo.

FIG. 2: Expression of αBcrystallin in human articular chondrocytes isolated from healthy and OA-cartilage. Upper panel: Results of the densitometric analysis of the western blots, showing the mean expression (±SEM) in each group (relative to NoNo expression levels). *: Denotes statistical significance (p<0.05; Mann-Whitney U-test) compared to NoNo. Lower panel: Comparison of αBcrystallin expression at the mRNA level in chondrocytes isolated from 6 OA-patients. In 5 of 6 samples a marked reduction of αBcrystallin expression in OAOA samples was detected. mRNA levels were determined by quantitative real-time PCR. Expression levels have been calculated as the mean expression compared to 3 different household genes (GAPDH, HPRT and PPIA). Different signs represent the relative expression of each individual patient, normalized to OAOA samples.

FIG. 3: Relative expression of αBcrystallin as determined by Western blot of chondrocytes isolated from NoOA-cartilage and stimulated with pro-inflammatory cytokines. NoOA chondrocytes were isolated from 4 patients and stimulated with IL-1β (0.01 ng/ml-0.05 ng/ml) or TNF-α (10 ng/ml); OAOA chondrocytes were stimulated with IL-1β (0.01 ng/ml) or TNF-α (10 ng/ml). Each stimulation was performed in duplicate per patient. Bars represent the average of the relative expression of 4 patients ±SEM, Levels are normalized to unstimulated controls for NoOA and OAOA samples; * denotes statistical significance (p<0.05; Mann-Whitney U-test) compared to unstimulated controls. Black bars represent NoOA samples, white bars represent OAOA samples.

FIG. 4: Expression levels of CRYAB, Aggrecan, BMP-2 and COL2A1 as determined by real-time PCR. Expression of CRYAB was suppressed by 21-mer siRNA sequences (siCRYAB). Non-silencing siRNA (Negative siControl) was used as a control. Expression levels have been calculated as the mean expression compared to 3 different household genes (GAPDH, HPRT and PPIA) in 4 different patients, 48 hours after transfection. Bars represent the average relative expression ±SEM, normalized to Negative siControl.

FIG. 5: Densitometric analysis of Western blots reveals a differential expression of HSP27 between the sample groups. Upper panel: Results of the densitometric analysis, showing the mean expression (±SEM) in each group (normalized to NoNo expression levels). P-values were calculated using Mann-Whitney U-test for the comparison between NoNo vs NoOA and NoNo vs OAOA. NoOA and OAOA samples were compared by Wilcoxon's paired sample test. Black bars: NoNo; white: NoOA; shaded: OAOA. Lower panel: Results of the densitometric analysis, showing the expression levels of HSP27 in chondrocytes isolated from visually intact zones (white bars) and visually damaged zones (shaded bars) of the same knee joint from 6 different patients.

FIG. 6: Relative expression of CRYAB and known differentiation markers were analyzed during dedifferentiation of chondrocytes seeded in monolayer at low density. The different curves represent the expression of CRYAB, BMP-2, COL2A1 at 0, 24, 48, 72 and 144, 192, 312, 384 and 456 hours. Expression levels are determined by real-time RT-PCR (CRYAB, BMP-2, COL2A1). Time points represent the average relative expression levels (to reference time point 0h), normalized to two household genes (GAPDH, PPIA) and represent the average of 5 different patient samples. In parallel, chondrocytes were cultured in alginate beads (“Beads CRYAB”). These cells shows a stable expression of CRYAB.

FIG. 7: Relative expression of known differentiation markers at different time points in expanding chondrocyte cultures and in redifferentiated chondrocyte cultures. Phenotypically stable chondrocytes were seeded in monolayer at time-point 0 hours. After an initial period of expansion (48 h, 96 h, 120 h or 192 h) part of the cells were embedded in alginate beads for 1 week in an attempt to restore the original phenotype, characterized by high COL2A1 and high BMP-2 expression. After 1 week of alginate culture, mRNA levels of COL2A1 and BMP-2 were analyzed (solid and dotted lines, respectively). As might be expected, the monolayer expansion is characterized by a quick decrease in COL2A1 and BMP2 expression. Upon alginate encapsulation, it is clear that only those cultures, expanded for 48 hours, were capable to restore the original phenotype.

FIG. 8: Expression of HSP27 protein in chondrocytes seeded in monolayer at low density at different time points (0 hours, 48 h, Confluence Point 1, Confluence Point 2), as previously described. The different figures represent two different patient samples. Expression levels were determined by Western Blot as previously described. A time dependent increase was observed, which is associated with a loss of the chondrocyte phenotype.

FIG. 9: HSP27 protein levels in chondrocyte monolayer cultures were quantified by Western Blotting at different time points. Time points represent the average (±SEM) of at least 3 different patients.

### DESCRIPTION OF THE INVENTION

As already mentioned hereinbefore, the present invention is based on the observation that both HSP27 and CRYAB are early markers in the dedifferentiation of a culture of isolated pluripotent cells and/or chondrocytes.

It is accordingly a first objective of the present invention to provide the use of CRYAB, HSP27 or of HSP27 and CRYAB as markers in methods to monitor and assess the chondrogenic capacity of such a population of isolated cells, i.e. the capacity of said cells to give rise to or to form cartilage.

“CRYAB” or αBcrystallin (HSPb5) and “HSP27” (HSPb1) belong to the family of small heat shock proteins (sHSPs), which are a family of proteins with molecular weights below 30 kDa, whose function is to protect cells against stress factors. The expression of sHSPs may be induced by various stresses, such as heat-shock, oxidative stress and chemical stresses. These molecular chaperones have been implicated in many different cellular processes (reviewed in [4]). Based on its ability to interfere with diverse cellular events, it is not surprising that mutations in small heat shock protein genes are implicated in different diseases. For example, mutations in the CRYAB gene are associated with the neuromuscular disorder desmin-related myopathy [5] or with cataract [6]. Furthermore, this family of proteins have been related with cancer, Alzheimer, Parkinson and other neurodegenerative diseases (reviewed in [7]).

As used herein the “CRYAB” polypeptide is meant to be a protein encoded by a mammalian CRYAB gene, including allelic variants as well as biologically active fragments thereof containing conservative or non-conservative changes as well as artificial proteins that are substantially identical, i.e. 70%, 75%, 80%, 85%, 87%, 89%, 90%, 92%, 93%, 94%, 95%, 96%, 97%, 98%, or 99% identical to any one of the aforementioned CRYAB polypeptides. In a particular embodiment the CRYAB polypeptide is 70%, 75%, 80%, 85%, 87%, 89%, 90%, 92%, 93%, 94%, 95%, 96%, 97%, 98%, or 99% identical to the human CRYAB (encoded by Genbank Accession N° NM_{—}001885).

By analogy, the “CRYAB” polynucleotide is meant to include allelic variants as well as biologically active fragments thereof containing conservative or non-conservative changes as well as any nucleic acid molecule that is substantially identical, i.e. 70%, 75%, 80%, 85%, 87%, 89%, 90%, 92%, 93%, 94%, 95%, 96%, 97%, 98%, or 99% identical to any one of the aforementioned CRYAB encoding polynucleotides. In a particular embodiment the CRYAB polynucleotide is 70%, 75%, 80%, 85%, 87%, 89%, 90%, 92%, 93%, 94%, 95%, 96%, 97%, 98%, or 99% identical to the nucleic acid molecule encoding for human CRYAB (Genbank Accession N° NM_{—}001885).

As used herein the “HSP27” polypeptide is meant to be a protein encoded by a mammalian HSP27 gene, including allelic variants as well as biologically active fragments thereof containing conservative or non-conservative changes as well as artificial proteins that are substantially identical, i.e. 70%, 75%, 80%, 85%, 87%, 89%, 90%, 92%, 93%, 94%, 95%, 96%, 97%, 98%, or 99% identical to any one of the aforementioned HSP27 polypeptides. In a particular embodiment the HSP27 polypeptide is 70%, 75%, 80%, 85%, 87%, 89%, 90%, 92%, 93%, 94%, 95%, 96%, 97%, 98%, or 99% identical to the human HSP27 (encoded by Genbank Accession N° NM_{—}001540).

By analogy, the “HSP27” polynucleotide is meant to include allelic variants as well as biologically active fragments thereof containing conservative or non-conservative changes as well as any nucleic acid molecule that is substantially identical, i.e. 70%, 75%, 80%, 85%, 87%, 89%, 90%, 92%, 93%, 94%, 95%, 96%, 97%, 98%, or 99% identical to any one of the aforementioned HSP27 encoding polynucleotides. In a particular embodiment the HSP27 plynucleotide is 70%, 75%, 80%, 85%, 87%, 89%, 90%, 92%, 93%, 94%, 95%, 96%, 97%, 98%, or 99% identical to the nucleic acid molecule encoding for human HSP27 (Genbank Accession N° NM_{—}001540).

As used herein the “COL2A1” polypeptide is meant to be a protein encoded by a mammalian COL2A1 gene, including allelic variants as well as biologically active fragments thereof containing conservative or non-conservative changes as well as artificial proteins that are substantially identical, i.e. 70%, 75%, 80%, 85%, 87%, 89%, 90%, 92%, 93%, 94%, 95%, 96%, 97%, 98%, or 99% identical to any one of the aforementioned COL2A1 polypeptides. In a particular embodiment the COL2A1 polypeptide is 70%, 75%, 80%, 85%, 87%, 89%, 90%, 92%, 93%, 94%, 95%, (CO, 97%, 98%, or 99% identical to the human COL2A1 (encoded by Genbank Accession N° NM_{—}001844.4).

By analogy, the “COL2A1” polynucleotide is meant to include allelic variants as well as biologically active fragments thereof containing conservative or non-conservative changes as well as any nucleic acid molecule that is substantially identical, i.e. 70%, 75%, 80%, 85%, 87%, 89%, 90%, 92%, 93%, 94%, 95%, 96%, 97%, 98%, or 99% identical to any one of the aforementioned COL2A1 encoding polynucleotides. In a particular embodiment the COL2A1 polynucleotide is 70%, 75%, 80%, 85%, 87%, 89%, 90%, 92%, 93%, 94%, 95%, 96%, 97%, 98%, or 99% identical to the nucleic acid molecule encoding for human COL2A1 (Genbank Accession N° NM_{—}001844.4).

As used herein, the terms “polynucleotide” and “nucleic acid” are used interchangeably to refer polynucleotides of any length, either deoxyribonucleotides or ribonucleotides or analogs (e.g., inosine, 7-deazaguanosine, etc.) thereof. “Oligonucleotides” refer to polynucleotides of less than 100 nucleotides in length, preferably less than 50 nucleotides in length, and most preferably about 10-30 nucleotides in length. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can include modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

“Polypeptide” refers to any peptide or protein comprising amino acids joined to each other by peptide bonds or modified peptide bonds. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature.

Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications (see, for instance, Proteins-Structure and Molecular Properties, 2nd Ed., T. E. Creighton, W. H. Freeman and Company, New York, 1993; Wold, F., Post-translational Protein Modifications: Perspectives and Prospects, pgs. 1-12 in Postranslational Covalent Modification of Proteins, B. C. Johnson, Ed., Academic Press, New York, 1983; Seifter et al., “Analysis for protein modifications and nonprotein cofactors”, Meth Enzymol (1990) 182: 626-646 and Rattan et al., “Protein Synthesis: Post-translational Modifications and Aging”, Ann NY Acad Sci (1992) 663: 4842).

### Sequence Identity

The percentage identity of nucleic acid and polypeptide sequences can be calculated using commercially available algorithms, which compare a reference sequence with a query sequence. The following programs (provided by the National Center for Biotechnology Information) may be used to determine homologies/identities: BLAST, gapped BLAST, BLASTN and PSI-BLAST, which may be used with default parameters.

The algorithm GAP (Genetics Computer Group, Madison, Wis.) uses the Needleman and Wunsch algorithm to align two complete sequences that maximizes the number of matches and minimizes the number of gaps. Generally, the default parameters are used, with a gap creation penalty=12 and gap extension penalty=4.

Another method for determining the best overall match between a nucleic acid sequence or a portion thereof, and a query sequence is the use of the FASTDB computer program based on the algorithm of Brutlag et al (Comp. App. Biosci., 6; 237-245 (1990)). The program provides a global sequence alignment. The result of said global sequence alignment is in percent identity. Suitable parameters used in a FASTDB search of a DNA sequence to calculate percent identity are: Matrix=Unitary, k-tuple=4, Mismatch penalty=1, Joining Penalty=30, Randomization Group Length=0, Cutoff Score=1, Gap Penalty=5, Gap Size Penalty=0.05, and Window Size=500 or query sequence length in nucleotide bases, whichever is shorter. Suitable parameters to calculate percent identity and similarity of an amino acid alignment are: Matrix=PAM 150, k-tuple=2, Mismatch Penalty=1, Joining Penalty=20, Randomization Group Length=0, Cutoff Score=1, Gap Penalty=5, Gap Size Penalty=0.05, and Window Size=500 or query sequence length in nucleotide bases, whichever is shorter.

### CRYAB, HSP27 and Optional COL2A1 Expression

The methods of the present invention to monitor and assess the chondrogenic capacity of a cell culture (herein also referred to as cell population or population of cells) include determining the expression levels of at least CRYAB or HSP27 in said cells, and alternatively of CRYAB and HSP27 at regular time intervals. In a further embodiment the methods of the present invention include determining the expression levels of CRYAB with at least the expression levels of HSP27 or COL2A1 at regular time intervals. In an even further embodiment the methods include determining the expression levels of CRYAB, HSP27 and COL2A1 at regular time intervals.

The “expression” generally refers to the process by which polynucleotides are transcribed into mRNA and/or the process by which the mRNA is subsequently translated into peptides, polypeptides or proteins. Hence the “expression” of a gene product, in the present invention of CRYAB, HSP27 and COL2A1, can be determined either at the nucleic acid level or the protein level.

Detection can be by any appropriate method, including, e.g., detecting the quantity of mRNA transcribed from the gene or the quantity of nucleic acids derived from the mRNA transcripts. Examples of nucleic acids derived from an mRNA include a cDNA produced from the reverse transcription of the mRNA, an RNA transcribed from the cDNA, a DNA amplified from the cDNA, an RNA transcribed from the amplified cDNA, and the like. In order to detect the level of mRNA expression, the amount of the derived nucleic acid should be proportional to the amount of the mRNA transcript from which it is derived. The mRNA expression level of a gene can be detected by any method, including hybridization (e.g., nucleic acid arrays, Northern blot analysis, etc.) and/or amplification procedures according to methods widely known in the art. For example, the RNA in or from a sample can be detected directly or after amplification. Any suitable method of amplification may be used. In one embodiment, cDNA is reversed transcribed from RNA, and then optionally amplified, for example, by PCR. After amplification, the resulting DNA fragments can for example, be detected by agarose gel electrophoresis followed by visualization with ethidium bromide staining and ultraviolet illumination. A specific amplification of differentially expressed genes of interest can be verified by demonstrating that the amplified DNA fragment has the predicted size, exhibits the predicated restriction digestion pattern and/or hybridizes to the correct cloned DNA sequence.

In hybridization methods a probe, i.e. nucleic acid molecules having at least 10 nucleotides and exhibiting sequence complementarity or homology to the nucleic acid molecule to be determined, are used. It is known in the art that a “perfectly matched” probe is not needed for a specific hybridization. A probe useful for detecting mRNA is at least about 80%, 85%, 90%, 95%, 97% or 99% identical to the homologous region in the nucleic acid molecule to be determined. In one aspect, a probe is about 50 to about 75, nucleotides or, alternatively, about 50 to about 100 nucleotides in length. These probes can be designed from the sequence of full length genes. In certain embodiments, it will be advantageous to employ nucleic acid sequences as described herein in combination with an appropriate label for detecting hybridization and/or complementary sequences. A wide variety of appropriate labels, markers and/or reporters are known in the art, including fluorescent, radioactive, enzymatic or other ligands, such as avidin/biotin, which are capable of giving a detectable signal. One can employ a fluorescent label or an enzyme tag, such as urease, alkaline phosphatase or peroxidase, instead of radioactive or other environmental undesirable reagents. In the case of enzyme tags, colorimetric indicator substrates are known that can be employed to provide a signal that is visible to the human eye or spectrophotometrically, to identify specific hybridization with complementary nucleic acid-containing samples.

Detection of the level of gene expression can also include detecting the quantity of the polypeptide or protein encoded by the gene. A variety of techniques are available in the art for protein analysis. They include but are not limited to radioimmunoassay (RIA), ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays and PAGE-SDS. One method to determine protein level involves (a) providing a biological sample containing polypeptides; and (b) measuring the amount of any immunospecific binding that occurs between an antibody reactive to the expression product of a gene of interest and a component in the sample, in which the amount of immunospecific binding indicates the level of the expressed proteins. Antibodies that specifically recognize and bind to the protein products of these genes are required for these immunoassays. These may be purchased from commercial vendors or generated and screened using methods well known in the art. See e.g., Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (M J. MacPherson, B. D. Hames and G. R. Taylor eds. (1995)), Harlow and Lane, eds. (1988) ANTIBODIES, A LABORATORY MANUAL and ANIMAL CELL CULTURE (R. I. Freshney, ed. (1987)).

In order to determine a change in expression and to compare the expression of CRAYB, HSP27 and COL2A1, the expression levels determined using any one of the aforementioned methods are normalized vis-à-vis the expression of a household gene such as GAPDH, HPRT, PPIA, Actine and the ribosomal proteins L19 and L32, and compared to the expression at start of the cell culture (t0). In a particular embodiment the expression levels are determined at least twice, more in particular at least 3, 4, 5, 6, 7, 8, 9, or 10 times over the period required to double the cell count in the cell culture.

As will be apparent to the skilled artisan, alternatively the expression levels of CRYAB, HSP27 and COL2A1 are compared with the ‘control’ expression level(s) of said genes in chondrogenic stable cells. These ‘control’ expression levels typically consist of the mean expression level(s) of said genes as determined in a representative set of isolated pluripotent cells and/or chondrocytes; preferably said ‘control’ levels are predetermined. Thus, in a particular embodiment, the method comprises the step of comparing the expression levels of said genes with the predetermined (pre-established) control expression levels of said genes. The latter may be presented as a combined value on an incremental scale reflecting the chondrogenic capacity of isolated pluripotent cells and/or chondrocytes, using art known scoring models such as for described in WO2008061804: MARKER GENES FOR USE IN THE IDENTIFICATION OF CHONDROCYTE PHENOTYPIC STABILITY AND IN THE SCREENING OF FACTORS INFLUENCING CARTILAGE PRODUCTION. In said embodiment the methods to monitor and assess the phenotypic stability of a cell expansion culture of isolated pluripotent cells and/or chondrocytes, comprise the step of determining the expression levels of CRYAB, HSP27 or of CRYAB and HSP27, in said cells and comparing said expression level(s) with the predetermined (pre-established) control expression levels of said genes.

As is shown in the examples hereinafter, blocking CRYAB expression in an in vitro culture of isolated chondrocytes and the transient decrease in CRYAB expression in a dedifferentiating cell culture of isolated chondrocytes, is accompanied with a decreased expression of the chondrocyte specific genes BMP-2, aggrecan and collagen type II. Thus in one embodiment of the present invention, a transient decrease in CRYAB expression in the in vitro culture of isolated pluripotent cells and/or chondrocytes, is indicative for a reduction in chondrogenic capacity of said cells, wherein the lowest CRYAB expression within said transient change, marks the start of the phenotypic change of said cell culture. In particular when in combination with an increase in HSP27 expression.

When the cell culture of isolated pluripotent cells and/or chondrocytes cells is kept beyond the lowest CRYAB expression within the transient change described herein, the cells lose the capability of expressing the typical markers of the chondrogenic phenotype like COL2A1 and aggrecan, upon reconstitution in their endogenous environment.

Using the methods of the present invention, it is now possible to identify cells that retain their full chondrogenic phenotype, and that are suitable for the repair of connective tissue, including cartilage, in particular in the repair of cartilage degeneration associated with osteoarthritis.

### Cell Lines and Therapeutic Application

The cell populations identified or obtained using the methods of the present invention are clearly a further object of the present invention.

These cell lines consist of primary cell cultures of cells identified as having a stable phenotype and/or high chondrogenic capacity, using the methods of the present invention.

Cells that are cultured directly from an animal or person are known as “primary cells”. With the exception of some derived from tumours, most primary cell cultures have limited lifespan. After a certain number of population doublings cells undergo the process of senescence and stop dividing, while generally retaining viability. Methods for growing suspension and adhesion cultures of primary cells are known to the person skilled in the art, such as for example described in “General Techniques of Cell Culture”, Maureen A. Harrison and Ian F. Rae, Cambridge University Press 2007.

As used in the methods of the present invention, the “primary cells” are derived from skeletal tissue, including cartilage; such as for example hyaline articular knee cartilage. In a particular embodiment the isolated cells consist skeletal precursor cell populations, including pluripotent cells and/or chondrocytes derived from skeletal tissue, such as for example derived from hyaline cartilage or fibrocartilage, including fibroblasts and meniscal chondrocytes. Thus in an even further embodiment the culture of isolated cells as used herein, consists of a cell culture of isolated chondrocytes, in particular chondrocytes derived from skeletal tissue, such as for example derived from hyaline cartilage or fibrocartilage, an are particularly selected from the group consisting of hyaline knee cartilage chondrocytes, synovial fibroblasts and meniscal chondrocytes.

In a particular embodiment the isolated chondrocytes as used in the methods of the present invention are characterized in that they express CRYAB, i.e. the early chondrocyte differentiation marker as identified by the present invention, with low or even undetectable expression levels of the further chondrocyte differentiation markers like HSP27 and COL2A1. In a particular embodiment of the present invention, the isolated chondrocytes as used in the present invention, are characterized in that, they are positive for CRYAB and the conventional chondrocyte differentiation markers Aggrecan, BMP-2 and COL2A1 with relative low, to no expression of HSP27, ALK1 and collagen type I.

As already mentioned hereinbefore, using the methods of the present invention, one may identify culture conditions and/or treatments that enhance the phenotypic stability and/or chondrogenic capacity of the cell culture. Such a homogeneous cell population makes them good candidates for tissue engineering protocols. Tissue engineering protocols are known to the person skilled in the art and include for example the method of Brittberg M. et al. (N. Eng. J. Med. 1994 331:889-895) wherein the population of cells obtained using the methods of the present invention are injected under a periosteal flap sutured to cover the cartilage defect. These protocols may further include a treatment with factors that stimulate the differentiation of the injected cells, such as Transforming Growth Factors-β (TGF-b's)

Based on the finding that CRYAB is a potential mediator of matrix gene expression in chondrocytes, in one embodiment of the present invention, the treatment would consist of a method to increase CRYAB expression in said cells. CRYAB positive cell populations with low or undetectable HSP27 expression levels are accordingly, a particular embodiment of the present invention.

Methods to increase expression of a gene of interest are known to the person skilled in the are and include genetic modification of the cells (e.g., by homologous recombination) by replacing, in whole or in part, the naturally occurring CRYAB promoter with all or part of a heterologous promoter, so that the cells express CRYAB polypeptide at higher levels. The heterologous promoter is inserted in such a manner that it is operatively linked to endogenous CRYAB encoding sequences. [See, for example, PCT International Publication No. WO 94/12650, PCT International Publication No. WO 92/20808, and PCT International Publication No. WO 91/09955.] It is also contemplated that, in addition to heterologous promoter DNA, amplifiable marker DNA (e.g., ada, dhfr, and the multifunctional cad gene, which encodes for carbamyl phosphate synthase, aspartate transcarbamylase, and dihydroorotase) and/or intron DNA may be inserted along with the heterologous promoter DNA. If linked to the CRYAB coding sequence, amplification of the marker DNA by standard selection methods results in co-amplification of the CRYAB coding sequences in the cells.

Thus in a further embodiment the CRYAB positive cell populations with low or undetectable HSP27 expression levels, are characterized in that the CRYAB positive cells are genetically modified cells using any one of the methods above.

### Assays and Therapeutic Application

Based on the above, it is also an object of the present invention to provide methods to identify compounds, or cell culture conditions capable of enhancing the phenotypic stability of a cell, said method comprising; applying the methods according to the invention in the presence and absence of the compound or culture condition to be tested, and determine whether said compound or cell culture condition is capable to prevent and/or delay the transient change in CRYAB expression. In a particular embodiment the present invention provides a method to identify compounds or cell culture conditions capable of enhancing the chondrogenic capacity of a cell, said method comprising applying the methods according to the invention in the presence and absence of the compound or cell culture condition to be tested, and determine whether said compound or cell culture condition is capable to prevent and/or delay the transient decrease in CRYAB expression, i.e. to extend the point up to which a cell expansion culture of said cells is identified as a phenotypic stable cell culture.

As already mentioned hereinbefore, and in a particular embodiment, the point up to which a cell expansion culture of said cells is identified as a phenotypic stable cell culture is characterized in;
a decrease in CRYAB expression down to about 70% or less of the initial CRYAB expression; in particular down to about 60% to about 40% of the initial CRYAB expression; and
an increase in HSP27 expression up to about 140% or more of the initial HSP27 expression; in particular up to about 150% to about 250% of the initial HSP27 expression.

“Compounds” as used includes, but is not limited to; small molecules including both organic and inorganic molecules with a molecular weight of less than 2000 daltons; proteins; peptides; antisense oligonucleotides; siRNAs; antibodies, including both polyclonal and monoclonal antibodies; ribozymes; etc.

In an alternative method to identify compounds or cell culture conditions capable of enhancing the chondrogenic capacity of a cell, said method comprises;
determine the expression levels of CRYAB and HSP27 at regular time intervals in a cell culture of isolated pluripotent cells and/or chondrocytes, both in the presence and absence of the compound or cell culture condition to be tested;
   wherein a compound or cell culture condition capable to prevent an increase in HSP27 expression and to delay or prevent the transient decrease in CRYAB expression, is identified as a compound or cell culture condition that enhances the chondrogenic capacity of a cell.

In the aforementioned method, the combined increase in HSP27 expression and the transient decrease in CRYAB expression, and more in particular the lowest CRYAB expression within said transient decrease, mark the point up to which a cell culture of said cells is identified as a phenotypic stable cell culture. The particular parameters associated with said point, like a decrease in CRYAB expression of at least 30% and an increase of HSP27 of at least 150% are already provided in more detail herein before and applicable in the aforementioned method of identifying compounds (treatments) or cell culture conditions that are capable of enhancing the phenotypic stability of the isolated cells according to the present invention.

“Cell culture conditions” such as for example addition of growth factors, a particular medium or cell culture conditions, such as for example non-adherent growth conditions, may influence the phenotypic stability of the cell culture. It is for example known that the phenotypic stability of cell cultures of isolated pluripotent cells and/or chondrocytes is sensitive for pro-inflamatory cytokines such as IL-1, IL-17, IL-18, IL-6, IL-8 or TNF, in particular IL-1 or TNF-α. Again, using the aforementioned methods of the present invention, it is now possible to identify compounds (treatments) or cell culture conditions that attenuate the sensitivity of isolated pluripotent cells and/or chondrocytes for pro-inflamatory cytokines.

Thus in a particular embodiment the present invention provides a method to identify compounds or cell culture conditions that attenuate the sensitivity of isolated pluripotent cells and/or chondrocytes for pro-inflammatory cytokines, said method comprising;
pre-treating said cells with one or more pro-inflammatory cytokines;
determine the expression levels of CRYAB and HSP27 at regular time intervals in a cell culture of said isolated and pretreated pluripotent cells and/or chondrocytes, both in the presence and absence of the compound or cell culture condition to be tested;
   wherein a compound or cell culture condition capable to prevent an increase in HSP27 expression and to delay or prevent the transient decrease in CRYAB expression, is identified as a compound or cell culture condition that attenuates the sensitivity of isolated pluripotent cells and/or chondrocytes for pro-inflammatory cytokines.

The pro-inflammatory cytokines as used herein are selected from the group consisting of IL-1, IL-17, IL-18, IL-6, IL-8 and TNF, in particular IL-1 or TNF-α.

In a further embodiment of the present invention, the assays are used to identify specific markers co-expressed with CRYAB and/or HSP27 and linked to the phenotypic stability of the cells, in particular cell surface markers that can be used for sorting of a heterogeneous cell pool into a subpopulation of phenotypically stable cells.

Conventional cell sorting methods (e.g., fluorescence-activated cell sorting (FACS)) can be used to isolate those cells in which the cell surface marker, co-detectable with CRYAB and/or HSP27, is expressed. A fluorescently-labelled antibody is then used to specifically bind a cell surface polypeptide used as the heterologous marker. Alternatively, an unlabeled antibody can be use to specifically bind the cell surface polypeptide, and a second, labeled antibody can then be used to specifically bind the first antibody. The fluorescently-tagged precursor cells can then be sorted away from other cells in the sample by FACS, for example. Other techniques, such as the use of protein-conjugated magnetic beads that selectively bind particular cells, can also be used. Suitable kits are commercially available. Generally, such kits utilize a tagged antibody (e.g., a biotintagged antibody) to bind the cell surface marker protein. The antibody-bound cells are contacted with a magnetic bead-protein conjugate, where the protein portion of the bead-protein conjugate specifically binds the tagged antibody. For example, a streptavidin-magnetic bead conjugate can be used to bind a biotin-tagged antibody to produce a complex containing the magnetic bead-protein conjugate, the tagged antibody, and the cell expressing the marker protein. Such complexes can be separated from other cells by temporarily adhering the complex to a magnet and separating the adhered cells from the other cells (i.e., a population of cells depleted for, e.g., skeletal precursor cells). Magnetic beads that are covalently coupled to a secondary antibody are commercially available. Other antibody-based methods for sorting cells, like the use of affinity chromatography or the retaining of cells expressing the particular cell surface proteins via Petri dishes coated with antibodies directed against the latter, also are known in the art and can be used in the invention.

It is accordingly an object of the present invention, to provide a method to identify markers co-expressed with CRYAB, said method comprising a gene expression analysis of cells that experience a transient change in CRYAB expression, wherein genes whose expression mimics the transient change or are only detectable at the maximal respectively minimal expression of CRYAB during the transient change, are identified as markers co-expressed with CRYAB.

The gene expression analysis, can be done using any technology that allows to determine the expression of a plurality of genes over time, such as semi-quantitative RT-PCR, by Northern hybridization or by DNA arrays or DNA chips.

In a further objective, the present invention provides the use of the markers co-expressed with CRYAB, in a method of sorting and enriching a cell culture of isolated pluripotent cells and/or chondrocytes, with phenotypic stable cells.

In a final embodiment the present invention provides the cells defined and obtainable using any one of the methods mentioned hereinbefore or the compounds identified using the methods of the present invention, for use as a medicine, in particular in the repair of connective tissue, more in particular in the repair of cartilage, even more in particular in the repair of cartilage degeneration associated with osteoarthritis.

In one aspect of this embodiment it includes pharmaceutical compositions comprising said cells. For example, tissue engineering protocols may include the application of bio-resorbable polymers (e.g. polylactic acid or polyglycolic avid) to fill the lesion. In such a use, a composition could contain a matrix seeded or mixed with cells of the present invention and eventually coated or mixed with further growth factors. In an alternative embodiment such a composition could consist of a prosthetic device useful in orthopedic reconstructive surgery, coated with cells of the present invention.

In another aspect it includes pharmaceutical compositions comprising the compounds identified using the methods of the present invention.

The pharmaceutical compositions of the present invention can be prepared by any known or otherwise effective method for formulating or manufacturing the selected product form. Methods for preparing the pharmaceutical compositions according to the present invention can be found in “Remington's Pharmaceutical Sciences”, Mid. Publishing Co., Easton, Pa., USA.

For example, the compounds can be formulated along with common excipients, diluents, or carriers, and formed into oral tablets, capsules, sprays, mouth washes, lozenges, treated substrates (e.g., oral or topical swabs, pads, or disposable, non-digestible substrate treated with the compositions of the present invention); oral liquids (e.g., suspensions, solutions, emulsions), powders, or any other suitable dosage form.

Non-limiting examples of suitable excipients, diluents, and carriers can be found in “Handbook of Pharmaceutical Excipients”, Second edition, American Pharmaceutical Association, 1994 and include: fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; adsorptive carriers such as kaolin and bentonite; carriers such as propylene glycol and ethyl alcohol, and lubricants such as talc, calcium and magnesium stearate, and solid polyethyl glycols.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXAMPLES

The following examples illustrate the invention. Other embodiments will occur to the person skilled in the art in light of these examples.

### Example 1

### αBcrystallin, a Potential Mediator of Matrix Gene Expression in Chondrocytes During the Development of Osteoarthritis

### 1.1 Materials and Methods

### 2-DE Analysis

In a preliminary study, protein extracts of chondrocytes (6 NoNo, 7 NoOA and 7 OAOA samples) were analyzed by a 2-DE approach, as described in [8]. Briefly, soluble and hydrophobic fractions of protein extracts of chondrocytes were separated by 2-DE. Sypro Ruby stained gels were scanned and analyzed using PDQuest V 7.1. Statistically significant differentially expressed spots (p<0.05; Mann-Whitney U-test) were excised and subjected to tandem mass spectrometry for identification.

### Isolation of Chondrocytes

Human articular chondrocytes were isolated as previously described. Articular knee cartilage from donors without arthropathy (NoNo) (3 male, 2 female, mean age: 44±25 years) was obtained within 24 h post-mortem. All donors had died as a result of trauma or a brief illness and none of them had been receiving corticosteroids or cytostatic drugs. OA affected cartilage was obtained from patients (6 male, 11 female, mean ages: 62±11 years) within 24 h from total knee arthroplasty. The cartilage from each of these patients was separated in visually intact cartilage (NoOA) and cartilage showing OA-lesions (DADA). The study was approved by the local Ethics Committee. The cartilage obtained was diced into small fragments and chondrocytes were isolated by sequential enzymatic digestion (hyaluronidase, pronase, collagenase (Sigma-Aldrich, Steinheim, Germany)) as described in detail elsewhere [9]. Trypan blue exclusion revealed that >95% of the cells were viable after isolation.

### Culture of Chondrocytes in Alginate Gel

Chondrocyte cultures in alginate beads were prepared as described by Guo et al [10], with some modifications [11]. Briefly, chondrocytes suspended in 1 volume of double-concentrated Hanks' balanced salt solution (HBSS; Gibco, Grand Island, N.Y., USA) without calcium and magnesium, were carefully mixed with an equal volume of 2% alginate. The final chondrocyte concentration was 5×10⁶ cells/ml. The chondrocyte-alginate suspension was slowly dripped through a 23-gauge needle into a 102 mM solution of calcium chloride and the beads were allowed to polymerize for 10 minutes at room temperature. Calcium chloride was removed and the beads were washed 3 times with 0.9% sodium chloride. The beads were maintained in a 6-well plate (20 beads/well; ±50.000 chondroctyes/bead) containing DMEM (Gibco) with 10% fetal calf serum, antibiotics and antimycotics (Gibco) in an incubator at 37° C. and in 5% CO₂. Medium was replaced three times a week for 10 days. After the culture period, the medium was aspirated and the alginate beads were washed and dissolved by incubation in 55 mM tri-sodium citrate dihydrate pH 6.8, at room temperature. The resulting suspension was centrifuged at 1500 rpm for 10 min to separate cells with their CAM from the constituents of the interterritorial matrix. The resulting cell-pellet was washed three times with PBS.

### Western Blot Analysis

Cell lysates of chondrocytes were prepared by resuspending cell pellets in 40 mM Tris from the ReadyPrep Sequential Extraction Kit (Bio-Rad, Hercules, Calif., USA), containing protease inhibitors (Roche Diagnostics, Mannheim, Germany) and a phosphatase inhibitor-cocktail (Sigma-Aldrich, Steinheim, Germany). Cells were lysed by sonication and the soluble proteins were isolated by centrifugation. Equal amounts (30 μg as determined by 2-D Quant kit, GE Healthcare, Fairfield, USA) of soluble fractions of 13 samples (5 NoNo, 4 NoOA and 4 OAOA) were loaded on 10% SDS-PAGE gel. Equal loading was verified by Ponceau S staining (data not shown). MagicMark (Invitrogen, Paisley, UK) protein standards were run as molecular weight markers. Following 1-D gel electrophoresis, proteins were transferred to nitrocellulose membranes (Bio-Rad). The resulting membranes were immunostained with rabbit anti-αBcrystallin (FL-175) (Santa-Cruz, Biotechnology, Santa Cruz, USA) followed by a anti-rabbit HRP-conjugated secondary antibody (Pierce, Rockford, Ill., USA) and ECL chemiluminescence detection (Supersignal West Dura Extended Duration Substrate, Pierce). Chemiluminescence images were recorded using the VersaDoc-imaging system (Bio-Rad). Image analysis was performed by Quantity One v 4.4.0 (Bio-Rad).

### Immunofluorescence Microscopy

Chondrocytes of cartilage samples of 3 additional patients were isolated and cultured as described above. Chondrocytes, cultivated in alginate beads, were washed in PBS for 5 minutes. The medium used during sample processing was a modified Hanks Balanced Salt Solution (mHBSS). Chondrocytes were fixed in the alginate beads using 4% para-formaldehyde (Sigma-Aldrich) for 15 minutes. Fixation was stopped by 3 washes in 100 mM glycine. Alginate beads were smeared on poly-1-lysine coated glass slides. Specimens were permeabilized using Triton X-100 (Sigma-Aldrich) 0.5%. Samples were washed and blocked using 5% Normal Goat Serum (NGS) for 1 h and were incubated overnight at 4° C. with primary mouse monoclonal IgG₁ anti-αBcrystallin (Abcam, Cambridge, UK) in 1% NGS in mHBSS. After three washes, they were incubated with anti-mouse IgG₁ AlexaFluor 488-conjugated secondary antibody (Molecular Probes) in 1% NGS for 1 h. Isotype controls were prepared and analyzed in parallel as negative controls. Samples were washed three times and incubated in DAPI nuclear stain (Molecular Probes). Finally, glass slides were washed four times in mHBSS. The slides were mounted on a coverslip and samples were examined with an AxioVert fluorescence microscope equipped with an ApoTome-module (Carl Zeiss). This allows to visualize cells in a near confocal-like way. At least 20, randomly selected, individual chondrocytes of each sample were imaged.

Chondrocyte Culture with Pro-Inflammatory Cytokines

Chondrocytes were isolated from 4 additional patients (2 male, 2 female) and cultured as described above for 7 days with IL-1β (0.01 ng/ml; 0.05 ng/ml) or TNF-α (10 ng/ml). Each stimulation was performed in duplicate per patient. After the culture period cells were isolated, proteins extracted, separated and blotted as described above.

### Real-Time PCR

Chondrocytes from 6 additional patients (1 male, 5 female) were isolated and cultured as described above. After the culture period, Trizol (Invitrogen) was added to the isolated cells, and RNA was extracted according to the manufacturer's instructions, followed by an additional purification step (RNeasy mini-kit (Qiagen)). This step included the digestion of DNA by deoxyribonuclease I (Invitrogen). cDNA was synthesized with oligo(dT) primers using the Superscript kit (Invitrogen).

Real-time PCR was performed using the ABI 7000 Sequence Detection System (Applied Biosystems). Each reaction utilized 20 μl of iTaq Supermix with Rox (Bio-Rad, Hercules, Calif.) and 5 μl of cDNA and was performed in triplicate. The thermocycler conditions were 2 min at 50° C., followed by 2 min at 95° C. and 45 cycles, each at 95° C. for 15 s and 60° C. for 1 min. Expression levels were normalized to those of human GAPDH, HPRT and PPIA. Relative quantitation was calculated using the 2^{-ΔΔCt} method [12,13].

### RNAi

Chondrocytes, isolated from visually intact cartilage of OA-patients (NoOA), were cultured in alginate as described above. Experiments were performed on 4 different patient samples. After the culture period, cells were isolated and seeded in monolayer culture at a density of 50.000 cells/cm². Cells were transfected with 5 nM siRNA (final concentration) directed against the αBcrystallin mRNA (Qiagen, Venlo, The Netherlands) or with a non-silencing control (All Stars Negative control siRNA, Qiagen), using HiPerfect transfection reagent at a final concentration of 0.5% (Qiagen). Separate transfections using siRNA directed against different sequences in the αBcrystallin mRNA strand have been performed. The transcript sequences targeted by the different siRNA sequences were: 5′-CAGGTTCTCTGTCAACCTGGA-3′; 5′-CTCCAGGGAGTTCCACAGGAA-3′ and 5′-CAGGCCCAAATTATCAAGCTA-3 Cells were harvested at 48 hours and cDNA was synthesized as described above. Cell viability after transfection was at least 95% as determined by trypan blue exclusion.

### 1.2 Results

### Differential Proteome Analysis of Healthy and OA Chondrocytes Shows the Differential Expression of a Protein Identified as αBcrystallin

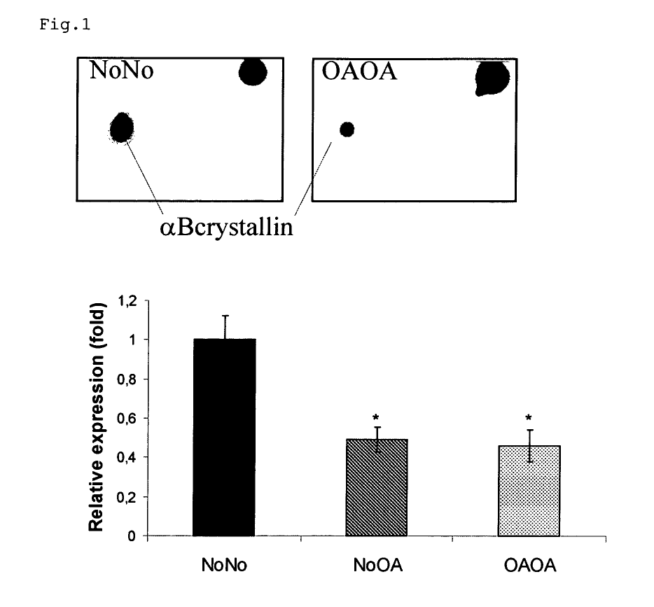

In a previous study, a two-dimensional gel-based differential proteome analysis was performed [8]. Protein expression patterns of chondrocytes isolated from 7 healthy patients (NoNo) and 6 OA-patients (visually intact (NoOA) and visually damaged zones (OAOA)) were compared to each other. This revealed the differential expression of a spot identified as αBcrystallin between normal and OA chondrocytes. This spot showed a down-regulation in NoOA samples (0.50 fold, p=0.007) and OAOA samples (0.46 fold, p=0.015) compared to healthy chondrocytes (FIG. 1).

### Western Blot Analysis Confirms Differential Abundance of αBcrystallin, a Protein Localized in the Cytoplasm

Expression levels of αBcrystallin were compared in lysates of chondrocytes isolated from healthy and OA-cartilage. Proteins extracted from chondrocytes isolated from healthy cartilage (NoNo, 5 patients) and visually intact NoOA and visually damaged (OAOA) OA-cartilage (4 patients) were separated by 1-D gelelectrophoresis and electroblotted. Western blot analysis confirms the higher abundance of αBcrystallin in NoNo samples. There is a gradual decrease in the abundance of αBcrystallin from NoNo, over NoOA, to OAOA samples. In addition, each individual OA-patient shows a higher intensity for αBcrystallin in NoOA versus OAOA chondrocytes. Western blots have been performed on albumin depleted synovial fluids of 3 OA, 3 RA and 3 SpA patients (up to 100 μg of protein was loaded on gel) and αBcrystallin could not be detected in these samples (results not shown).

The cellular localisation of αBcrystallin was investigated by immunofluorescence microscopy and the most intense staining was observed in the cytoplasm (data not shown).

Real-Time RT-PCR Confirms the Differential Transcription of αBcrystallin at the mRNA Level

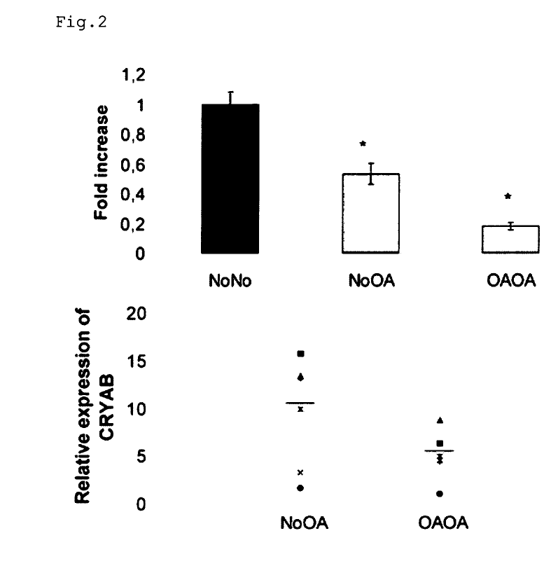

To evaluate whether the differential abundance of αBcrystallin is a consequence of a differential gene expression, mRNA levels for αBcrystallin were compared by real-time RT-PCR in 6 additional paired samples of NoOA and OAOA chondrocytes. Consistent with the results at the protein level (FIG. 2), the mRNA levels of αBcrystallin were upregulated in NoOA chondrocytes compared to OAOA chondrocytes in 5 of 6 samples analyzed (FIG. 2 lower panel).

The cytokines IL-1β and TNF-α Suppress the Expression of alphaBcrystallin

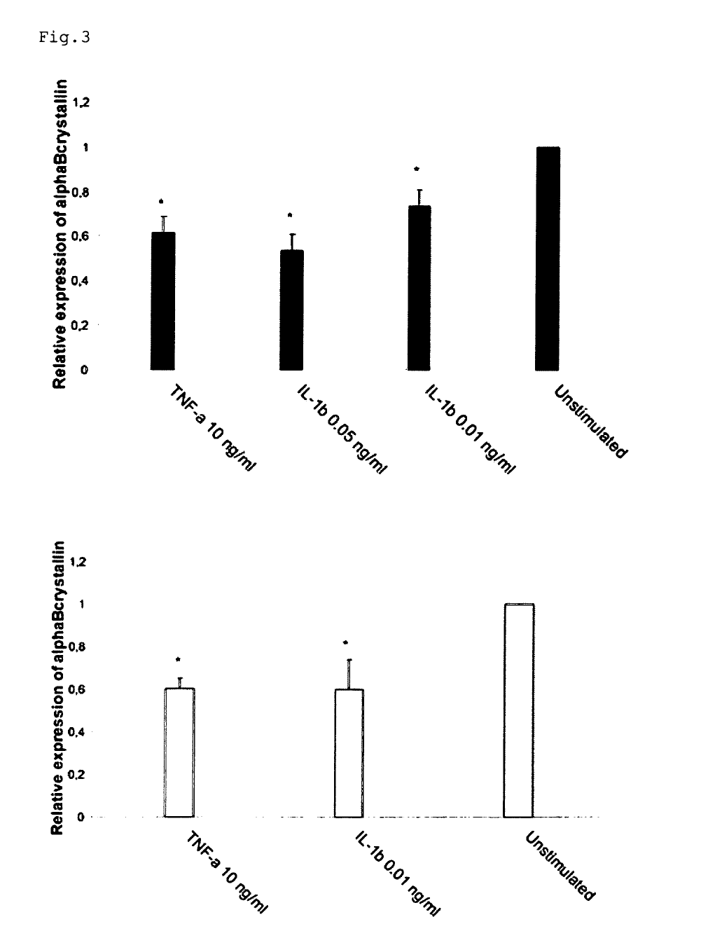

It is generally known that pro-inflammatory cytokines such as IL-1β and TNF-α modulate the chondrocyte's metabolism and play an important role in OA-pathogenesis. To determine if the expression of αBcrystallin is influenced by pro-inflammatory cytokines, chondrocytes isolated from NoOA and OAOA samples were treated with IL-1β and TNF-α. In both cases, the pro-inflammatory cytokines suppressed the expression of αBcrystallin. FIG. 3 shows the dose-dependent suppression of αBcrystallin expression by IL-1β. No significant differences were found between NoOA and OAOA chondrocytes in response to 0.1 ng/ml IL-1β and TNF-α.

Specific Knock-Down of αBcrystallin Expression by siRNA Results in Decreased Expression of Chondrocyte-Specific Markers

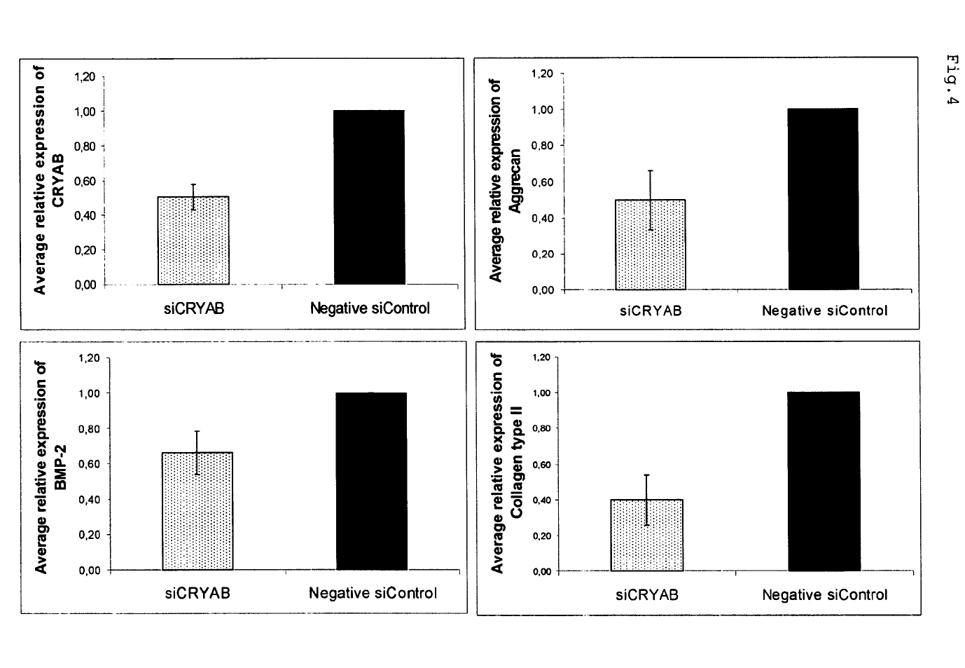

A siRNA sequence against αBcrystallin (siCRYAB) was transfected in cultured chondrocytes. Non-silencing siRNA was used as a negative control. As shown in FIG. 4, siRNA transfection resulted in a marked reduction of αBcrystallin expression in CRYAB-siRNA samples compared to negative controls. In parallel, a second sequence and third sequence were transfected, showing similar results (data not shown).

To explore the effects of a reduced expression of αBcrystallin on chondrocytes, the gene expression of 2 matrix genes (collagen type II and aggrecan) and the growth-factor, BMP-2, was compared between silenced and non-silenced chondrocytes by real-time PCR. FIG. 4 shows the average expression ±SEM (4 experiments) of collagen type II, aggrecan and BMP-2 in silenced and non-silenced samples. For the siCRYAB sequence, there is a marked reduction in gene expression for these genes compared to non-silenced controls. Samples transfected with a second and third sequence showed similar trends (data not shown).

Morphologic Dedifferentiation of Chondrocytes is Associated with a Changed Expression of αBcrystallin

Chondrocytes isolated from 2 OA-patients were seeded in monolayer cultures at low density (30.000 cells/cm²). As expected, this resulted in a quick morphologic change. At time of seeding, cells showed the typical rounded cell-shape associated with chondrocytes. After 144 hours almost all cells are attached to the surface and showed a fibroblast-like morphology. Moreover, near confluent cell cultures were observed at this time point. Relative expression of CRYAB, HSP-27 and known differentiation markers were analyzed during dedifferentiation of chondrocytes seeded in monolayer at low density. The different curves represent the expression of CRYAB, HSP27, BMP-2, COL2A1, Aggrecan, ALK-1 at 0, 24, 48, 72 and 144 hours. Expression levels are determined by western blotting (HSP27) or real-time RT-PCR (CRYAB, BMP-2, COL2A1, Aggrecan, ALK-1). Time points represent the average relative expression levels (to reference time point 0 h), normalized to two household genes (GAPDH, PPIA) and represent the average of 2 different patient samples.

### Example 2

### HSP27 in OA-Affected Chondrocytes

### 2.1 Materials and Methods

### Isolation and Culture of Chondrocytes

Human articular knee cartilage was obtained from 4 donors (one male, three female, median age ±SD: 70±10.9 years) after total knee arthroplasty. The study protocol was approved by the local Ethics Committee. The cartilage was diced into small fragments and chondrocytes were isolated by sequential enzymatic digestion (hyaluronidase, pronase, collagenase (Sigma-Aldrich, Steinheim, Germany)) as described in detail elsewhere [9]. Trypan blue exclusion revealed that >95% of the cells were viable after isolation.

Chondrocyte cultures in alginate beads were prepared and maintained as described previously [8]. For each sample, 15-17.5×10⁶ chondrocytes were lysed and treated as described below.

### Sample Preparation and Protein Digestion

Total cell lysate—Isolated chondrocytes were resuspended and lysed in a denaturing buffer containing 8 M urea. After reduction and alkylation, proteins were digested with sequencing grade trypsin at 37° C. for 12 to 16 h. Peptide solutions were desalted using solid-phase extraction cartridges (Oasis®, Waters, Milford, USA), dried in a vacuum-centrifuge and frozen at −80° C. until use.

Fractionation based on hydrophobicity and Mw—Isolated chondrocytes were resuspended in Reagent 1 (40 mM Tris) from the ReadyPrep Sequential Extraction Kit (Bio-Rad, Hercules, Calif., USA), containing protease inhibitors (Roche Diagnostics, Mannheim, Germany) and a phosphatase inhibitor-cocktail (Sigma-Aldrich, Steinheim, Germany). Cells were Lysed by Sonication and the Proteins were Extracted Using the ReadyPrep Sequential Extraction Kit (Bio-Rad) according to the manufacturer's protocol. This resulted in a soluble, an intermediate and a hydrophobic protein fraction [14]. All fractions were dialysed using 2 kDa Mw cut-off centrifugal devices (Vivaspin, Vivascience). The resulting concentrates were diluted in dissolution buffer (iTRAQ reagents kit, Applied Biosystems) supplemented with a SDS-containing denaturant solution. After reduction and alkylation, the intermediate and hydrophobic fractions were digested using sequencing grade trypsin. The soluble fraction was further fractionated, based on molecular weight using 300 kDa, 100 kDa, 10 kDa and 2 kDa Mw cut-off centrifugal devices (Vivaspin, Vivascience). Each resulting concentrate was digested as described above. For a second chondrocyte sample, the resulting pellet after the second extraction, was resuspended in a 2% SDS-solution containing 5% 3-mercaptoethanol. The suspension was heated at 95° C. for 5 minutes. The sample was cooled to room temperature and centrifuged at 15000 rpm. The resulting supernatans was subjected to 1-D gelelectrophoresis. Precision Plus All Blue standards (Bio-Rad) were run in parallel as molecular weight markers. The gel was stained with Colloidal Coomassie blue (Pierce). The lane was divided in eight zones and each zone was isolated and cut in smaller fragments followed by in-gel digestion as described previously [8], except that larger volumes were used.

On-Line Two-Dimensional nanoLC-MS

Peptides were resuspended in buffer A (Buffer A: 2.5 mM NaH₂PO₄, 3% ACN, pH=2.7). For ESI Qq-T of analysis the resuspended peptides were injected on an Ultimate 3000 autosampler (LC Packings, Sunnyvale, Calif., USA) and trapped on a Poros 10S strong-cation exchange (SCX) column (300 μm i.d.×15 cm). Peptides were eluted from the SCX column by a 840 min discontinuous gradient from 0 to 100% buffer B (Buffer B: 2.5 mM NaH₂PO₄, 500 mM NaCl, 3% ACN, pH=2.7, flow rate=6 μl/min). Two reversed-phase trap-columns (PepMap, LC Packings) were used for parallel trapping of the peptides, eluting from the SCX column, prior to the separation on a C18 PepMap column (LC Packings) by a 70 min linear gradient from 6 to 100% buffer C (80% acetonitrile and 0.1% formic acid in water). A Switchos pump (LC Packings) [pumping 12 μl/min buffer D (0.1% formic acid in water)] was coupled to the LC-system to wash the trap columns prior to peptide elution. ESI Qq-T of analysis was performed on a Q-T of Ultima mass spectrometer (Waters, Milford USA), which was coupled to the LC-system via a nano-LC inlet. The instrument was calibrated using fragment ions generated from MS/MS of Glu-fibrinopeptide B (Sigma-Aldrich).

### Database Searching

The Q-TOF was operated in a data-dependent mode by performing MS/MS scans for the 7 the most intense peaks from each MS scan. The MS scan range was 450-1100 m/z. Peak list files were generated from the raw data by Mascot Distiller version 2.1.0.0 (Matrix Science). The generated files were merged into a single .mgf file. Database searches against the Swiss-Prot database (version 51.6) were performed using an in-house licensed Mascot search engine (version 2.2.0.0). The search parameters were: a maximum of 1 missed cleavages using trypsin; fixed modifications were carbamidomethylation and variable modifications were oxidation of methionine and N-terminal acetylation; the mass tolerances were set to 0.20 and 0.15 Da for precursor ions and fragment ions respectively; one C13 was allowed. Only protein identifications with a p-value less than 0.01 were accepted. Protein identifications based on a single peptide hit were manually validated and only accepted if three consecutive y or b ions matched intense peaks in the MS/MS-spectrum.

### Western Blot Analysis

Chondrocyte samples of 6 healthy donors (4 male, 2 female, median age ±SD: 46.5±23.6 years) and 6 OA patients (3 male, 3 female, median age ±SD: 66.5±11.1 years) were collected, isolated and cultured as described previously. Cell lysates of chondrocytes were prepared by resuspending cell pellets in 40 mM Tris from the ReadyPrep Sequential Extraction Kit (Bio-Rad, Hercules, Calif., USA), containing protease inhibitors (Roche Diagnostics, Mannheim, Germany) and a phosphatase inhibitor-cocktail (Sigma-Aldrich, Steinheim, Germany). Cells were lysed by sonication and the soluble proteins were isolated by centrifugation. Equal amounts (30 μg as determined by 2-D Quant kit, GE Healthcare, Fairfield, USA) of soluble fractions of 18 samples [6 NoNo (chondrocytes isolated from healthy cartilage), 6 NoOA (chondrocytes isolated from visually intact cartilage of OA-patients) and 6 OAOA (chondrocytes isolated from visually damaged zones of OA-cartilage)] were loaded on 10% SDS-PAGE gels. Equal loading was verified by Ponceau S staining (data not shown). MagicMark (Invitrogen, Paisley, UK) protein standards were run as molecular weight markers. Following 1-D gel electrophoresis, proteins were transferred to nitrocellulose membranes (Bio-Rad). The resulting membranes were immunostained with a mouse monoclonal anti-HSP27 (G3.1) (Abcam, Cambridge, UK) followed by an anti-mouse HRP-conjugated secondary antibody (Pierce, Rockford, Ill., USA) and enhanced chemilumineschence detection (Supersignal West Dura Extended Duration Substrate, Pierce). Chemiluminescence images were recorded using the VersaDoc-imaging system (Bio-Rad). Image analysis was performed by Quantity One v 4.4.0 (Bio-Rad).

### 2.2. Results and Discussion

### Identification of Potential Chondrogenicity Markers

In a previous report we identified αBcrystallin as a potential interesting protein in OA pathogenesis (Example 1 above). αBcrystallin belongs to the family of small heat-shock proteins (sHSPs), a family of molecular chaperones with similar structures and functionalities [15]. Until now, 10 different sHSPs have been identified in human [16]. In our study, we identified three members of this family in the low molecular weight fraction: the previously described αBcrystallin (HSPb5), HSP20 (HSPb6) and HSP27 (HSPb1).

HSP27 and αBcrystallin are the most investigated sHSPs. Both are very closely related, in function as well as in structure (reviewed in [17]). Based hereon, we further examined the expression of HSP27 in OA-affected chondrocytes, revealing a reduced expression of this protein in cultured OA-chondrocytes. Our western blot data show a significant reduced expression of HSP27 in OA-chondrocytes compared to chondrocytes isolated from healthy cartilage (33% reduction, p<0.05-Mann Whitney U-test). In 5 of the 6 patients analysed, HSP27 shows a higher expression in chondrocytes isolated from visually intact compared to visually damaged zones from the same knee joint (FIG. 5). Chondrocytes were seeded in monolayer cultures at low density (30.000 cells/cm²). As expected, this resulted in a quick morphologic change. At time of seeding, cells showed the typical rounded cell-shape associated with chondrocytes. After 144 hours almost all cells are attached to the surface and showed a fibroblast-like morphology (data not shown). Moreover, near confluent cell cultures were observed at this time point. Cells were harvested at 3 different time points: 0 h, 72 h, 196 h. As determined by Western blotting, this morphologic dedifferentiation was associated with an increasing abundance of HSP27.

### Example 3

### Further Characterization of the End Point Marking the Phenotypic Change

### 3.1. Materials and Methods

### Real-Time PCR

After the culture period, Trizol (Invitrogen) was added to the isolated cells, and RNA was extracted according to the manufacturer's instructions, followed by an additional purification step (RNeasy mini-kit (Qiagen)). This step included the digestion of DNA by deoxyribonuclease I (Invitrogen). cDNA was synthesized with oligo(dT) primers using the Superscript kit (Invitrogen).

Real-time PCR was performed using the ABI 7000 Sequence Detection System (Applied Biosystems). Each reaction utilized 20 μl of iTaq Supermix with Rox (Bio-Rad, Hercules, Calif.) and 5 μl of cDNA and was performed in triplicate. The thermocycler conditions were 2 min at 50° C., followed by 2 min at 95° C. and 45 cycles, each at 95° C. for 15 s and 60° C. for 1 min. Expression levels were normalized to those of human GAPDH and PPIA. Relative quantitation was calculated using the 2^{-ΔΔCt} method.

### Monolayer Culture

Phenotypically stable chondrocytes isolated from 5 OA-patients (3 female, 2 male, mean ages: 63.6±8.26 years) were seeded in monolayer culture at low density (30.000 cells/cm²). At 5 different time points Trizol (Invitrogen) was added to the isolated cells, and RNA was extracted according to the manufacturer's instructions, followed by cDNA preparation as described above. Real-time PCR conditions were similar as above.

### Redifferentiation Cultures

Phenotypically stable chondrocytes were seeded in monolayer culture at low density (30.000 cells/cm²). At 4 different time points (48 h, 96 h, 120 h, 192 h) Trizol (Invitrogen) was added to the isolated cells, and RNA was extracted according to the manufacturer's instructions, followed by cDNA preparation as described above. At the same time points, part of the cells were embedded in alginate beads (as described above), in an attempt to restore the original chondrocyte phenotype, characterized by a COL2A 1 and BMP2 levels which are comparable with those of phenotypically stable chondrocytes (0 h). After 1 week of alginate culture, the cells were isolated and cDNA prepared as described previously.

### 3.2. Results

Dedifferentiation of chondrocytes is associated with a changed expression of αBcrystallin Chondrocytes isolated from 5 OA-patients were seeded in monolayer cultures at low density (30.000 cells/cm²). As expected, this resulted in a quick morphologic change. At time of seeding, cells showed the typical rounded cell-shape associated with chondrocytes. After 144 hours almost all cells are attached to the surface and showed a fibroblast-like morphology. Moreover, near confluent cell cultures were observed at this time point. Relative expression of CRYAB and known differentiation markers were analyzed during dedifferentiation of chondrocytes seeded in monolayer at low density. The different curves represent the expression of CRYAB, COL2A1, and BMP-2 at different time points. Time points represent the average relative expression levels (to reference time point 0 h), normalized to two household genes (GAPDH, PPIA) and represent the average of 5 different patient samples. As indicated, monolayer culture is associated with a quick loss of the chondrocyte phenotype (indicated by the time-dependent decrease of COL2A1 and BMP-2 expression). The expression of CRYAB is characterized by an initial 2-fold decrease, with a minimum at 48 hours, followed by a slight increase compared to initial levels, which remain relatively stable over time. Chondrocytes cultured in alginate beads remain a stable CRYAB expression (“CRYAB beads”, FIG. 1). This might be expected as it is generally known that chondrocyte-culture in an alginate matrix results in a preservation of the chondrocyte phenotype.

Redifferentiation Experiments Associate the Time Point of Minimal CRYAB Levels with the Ultimate Time Point of Chondrocyte Redifferentiation

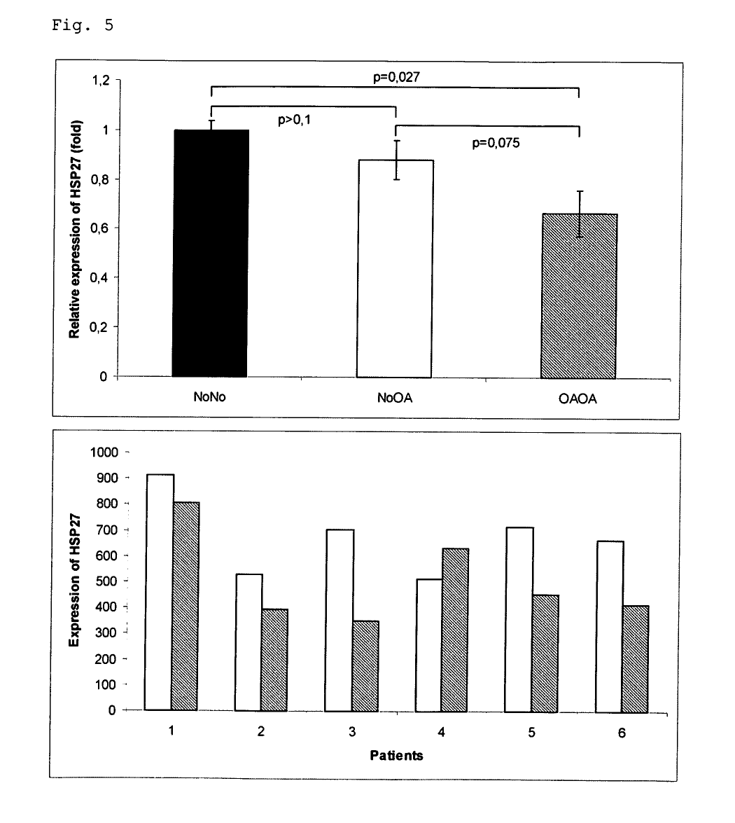

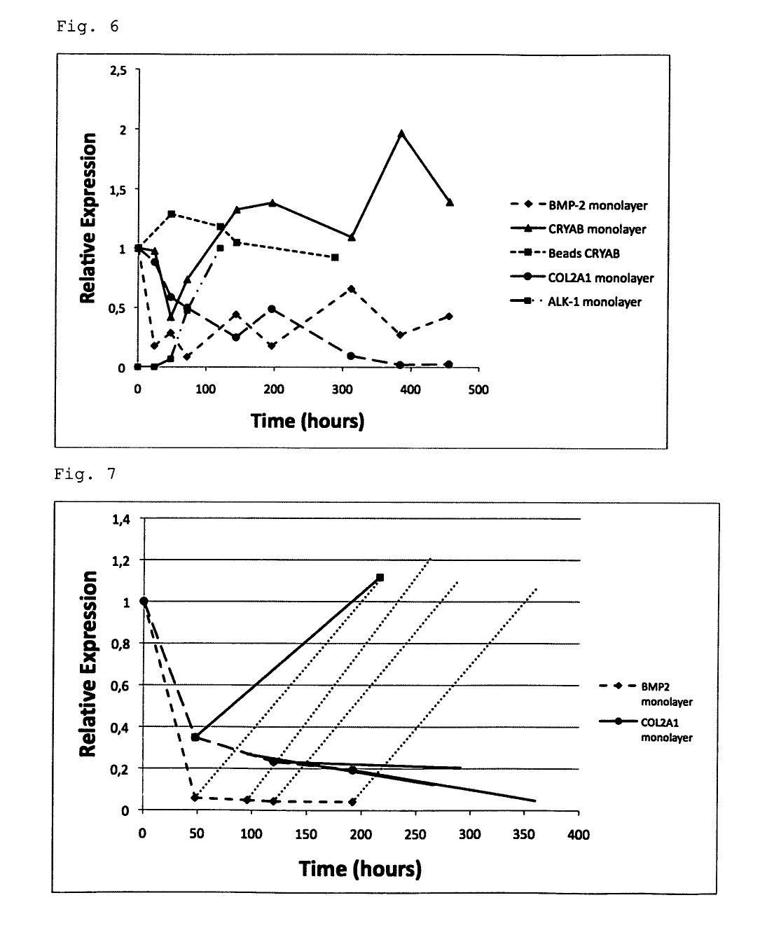

To confirm the association of minimal CRYAB-levels in expanding monolayer culture with a switch in chondrocyte phenotype, redifferentiation experiments were performed. Phenotypically stable chondrocytes were seeded in monolayer at time-point 0 hours. After an initial period of expansion (48 h, 96 h, 120 h or 192 h) part of the cells were embedded in alginate beads for 1 week in an attempt to restore the original phenotype, characterized by high COL2A1 and high BMP-2 expression. As indicated in FIG. 7, it is clear that only those cultures, expanded for 48 hours—the time point characterized by a minimal CRYAB expression in monolayer cultures (FIG. 6), were capable to restore the original phenotype (FIG. 7).

Dedifferentiation of Chondrocytes is Associated with an Increased Expression of HSP27

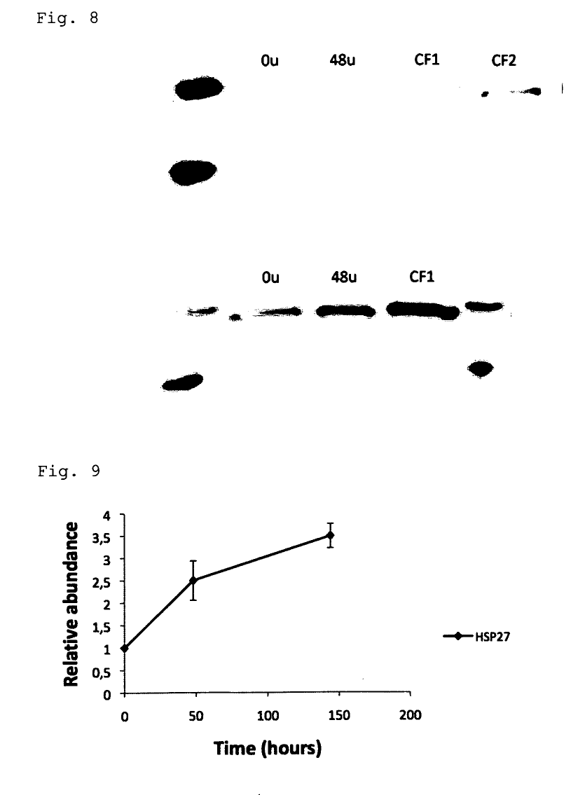

Chondrocytes isolated from OA-patients were seeded in monolayer cultures at low density (30.000 cells/cm²), as described above. At different time points, cells were lysed and proteins extracted. Proteins were separated, electroblotted and probed with anti-HSP27. FIG. 8 shows a time-dependent, and thereby dedifferentiation associated, increase in HSP27 protein expression levels. As shown in FIG. 9, at 48 hours—the ultimate observed time point that allows chondrocyte redifferentiation, the protein levels of HSP27 are more than double the initial expression. Further time points show a steadily increasing expression.

### Example 4

### Further Cell Types: Meniscus Chondrocytes

It is generally known, that articular chondrocytes, meniscus chondrocytes and synovial cells e.g. (synovial fibroblast) originate from a common precursor and show common functional properties. Articular and meniscus chondrocytes show common structural and functional properties, whereas synovial cells have been shown to have chondrogenic potential. Given their common ancestry, we expect for all of these cell types that CRYAB will act as an early chondrogenic marker, similar to the transient change observed in the hyaline knee cartilage chondrocytes.

Analogously to the hyaline knee cartilage chondrocytes, phenotypically stable meniscus chondrocytes isolated from human medial and/or lateral menisci will be seeded in monolayer culture to allow expansion of the cell population as described in: “Characterisation of human knee meniscus cell phenotype. Osteoarthritis Cartilage. 2005 July; 13(7):548-60.

### Meniscus Cell Isolation

Complete, visually intact human lateral and medial menisci are harvested 24 h postmortem in accordance with the currently accepted transplantation protocols and approved by the local ethics committee

Menisci are inspected macroscopically for gross degeneration. Anterior and posterior entheses and all synovial tissue are carefully trimmed by sharp dissection. The specimens are then diced into 1×1×1 mm pieces.

In order to obtain a single cell suspension these pieces are enzymatically digested for 4 to 6 h with 3 mg/ml collagenase grade II (Clostridium histolyticum, ICN Biomedicals Inc., Aurora, Ohio, USA) in Dulbecco's Modified Eagle's Medium (DMEM, GIBCO Invitrogen Co, Merelbeke, Belgium), supplemented with 10% fetal bovine serum (FBS, GIBCO Invitrogen Co), antibiotics and antimycotics (penicillin 10 U/ml, streptomycin 10 mg/ml, fungizone 0.025 mg/ml) (GIBCO Invitrogen Co), 0.002 M L-glutamine followed by the addition for 1-2 h of 2 mg/ml pronase (Streptomyces griseus pronase E, Merck, Darmstadt, Germany) in DMEM, anti-biotics and antimycotics, 0.002 M L-glutamine-16. Cells are washed, filtered and resuspended in Hank's balanced salt solution without Ca2 or Mg2 (HBSS, GIBCO Invitrogen Co) at the appropriate concentration depending on the culture conditions. Cells are counted and viability checked using trypan blue exclusion test.

Three-Dimensional Culture in Alginate Hydrogel

Meniscus cells suspended in one volume of HBSS are carefully mixed with an equal volume of 2% (weight/volume) alginate (low-viscosity alginate from Macrocystis pyrifera, Sigma-Aldrich, St Louis, Mo., USA) in HBSS (previously autoclaved for 15 min), obtaining a final concentration of 5×106 cells/ml in 1% alginate. Alginate beads are formed when this cell-alginate suspension is carefully dropped in a 102 mM CaCl₂ solution through a 23 Gauge needle. After a 10 min polymerization period, the beads are washed twice in 0.9% NaCl and then cultured in 5 ml of culture medium (DMEM, antibiotics, FBS 10%, L-glutamine, ascorbic acid 2-phosphate 50 mg/ml). The culture medium is typically changed three times a week. The cells are cultured in a 6-well plate (each well containing 30 beads, ±1.5×106 cells/well).

### Meniscus Cell Monolayer Culture

Cells are seeded in a T25 flask (Nunc, Roskilde, Denmark) at an initial density of 2×105 cells/cm2. The same culture medium is used as for the cells cultured in the alginate beads. Medium is changed three times a week.

When monolayer cultures become confluent after 5-7 days of culture, cells are washed with phosphate buffered saline without Ca2+ and Mg3+ (PBS, GIBCO Invitrogen Co) prior to treatment with trypsin-ethylenediaminetetraacetic acid (EDTA) (GIBCO Invitrogen Co) for 10 min. Culture medium with 10% FBS is used to neutralize the trypsin-EDTA solution. The cell suspension is centrifuged (1500 rpm, 10 min) and washed with culture medium. Cells are reseeded at an initial seeding density of 2×10⁴ cells/cm2.

At different time points cells will be isolated and RNA extracted as described above. Using Real-time PCR mRNA levels of the above mentioned markers (COL2A1, COL1A1, Aggrecan, CRYAB and HSP27) will be evaluated at the different time points. Moreover, at the different time points cells will be encapsulated in alginate beads as described above, in attempt to restore the original phenotype. The ability to express a similar marker profile upon encapsulation, compared to phenotypically stable chondrocytes, will be used to evaluate the phenotypical stability of the expanding cell culture. In a similar way the markers of the present invention will be used to evaluate the expansion—and thereby dedifferentiation—of synovial fibroblasts, another cell type isolated from joint tissue.

### Example 5

### Further Cell Types: Synovial Chondrocytes

Studies of the ontogenetic development of synovial joints have revealed that articular chondrocytes and synovial cells originate from a common precursor pooll and exist in a close functional relationship not only during fetal development but also in adult life. (“Enhancing and maintaining chondrogenesis of synovial fibroblasts by cartilage extracellular matrix protein matrilins”, Osteoarthritis and Cartilage (2008) 16, 1110-1117).

Furthermore, synovial cells have been shown to possess tremendous chondrogenic potential under various pathological conditions in vivo. For example, synovial chondromatosis is observed in disease states in which human chondroprogenitor cells of synovial origin sustain their high proliferative potential and capacity to differentiate into cells with chondrogenic character irrespective of the individual's age. Importantly, synovial cells share several properties with chondrocytes, including the production of extracellular matrix (ECM) proteins including cartilage oligomeric matrix protein (COMP), link proteins, and sulfated glycosaminoglycans (sGAG).

We accordingly expect that CRYAB will act as an early chondrogenic marker in synovial cells, similar to the transient change observed in the hyaline knee cartilage chondrocytes. Synovial fibroblasts will be isolated and expanded such as for example described in (“Enhancing and maintaining chondrogenesis of synovial fibroblasts by cartilage extracellular matrix protein matrilins”, Osteoarthritis and Cartilage (2008) 16, 1110-1117, and the markers of the present invention will be used to evaluate the expansion—and thereby dedifferentiation of said synovial fibroblasts.

### Harvest of Synovial Tissue

Each group of random biopsies of synovial tissue is obtained aseptically from the knee joints. The tissue is placed in cell culture medium at room temperature and subjected to tissue digestion within 2 h by art known procedures. Briefly, synovial tissue is finely minced, digested for 30 min at 37° C. in phosphate-buffered saline (PBS) containing 0.1% trypsin (Roche, Indianapolis, Ind., USA) and thereafter digested in 0.1% collagenase P (Roche, Indianapolis, Ind., USA) in DMEM/10% FBS for 2 h at 37° C. The cell suspension is then put through a 70 mm nylon filter and the cells are collected by centrifugation. Cells are kept in primary culture for 4 days (DMEM/10% FBS, 100 U/mL penicillin, 100 mg/mL streptomycin, including removal of non-adherent cells on days 2 and 4) and are subsequently used for SFB isolation.

### Negative Isolation of SFBs

Mixed populations of synovial cells (SCs) contain fibroblasts, monocytes, and macrophages. For negative isolation of SFBs from primary culture, adherent synovial cells are detached by short-term trypsinization for less than 1 min (0.25% trypsin/0.2% ethylenediaminetetraacetic acid EDTA, Gibco, Grand Island, N.Y., USA) and 107/mL SCs incubated with washed 4×107/mL Dynabeads M-450 CD14 (clone RM052; Dynal Biotech, Oslo, Norway) in PBS/2% FBS for 1 h at 4 C on an orbital shaker. Dynabeads CD14 are superparamagnetic polystyrene beads coated with a primary monoclonal antibody (mAb) specific for the CD14 membrane antigen predominantly expressed on monocytes and macrophages. PBS/2% FBS is then added to a final volume of 10 mL and the conjugated cells (monocytes and macrophages) and the unbound Dynabeads are collected using the Dynal Magnetic Particle Concentrator (Dynal Biotech, Oslo, Norway). The depleted supernatant with SFBs is transferred to a new tube for further study.

### 2D Expansion Culture of Isolated SFBs

SFBs are plated in DMEM containing 10% FBS at 1.8×106 cells/25 cm2 tissue culture flask. When monolayer cultures become confluent after 4-7 days of culture, cells are washed with phosphate buffered saline without Ca2+ and Mg2+ (PBS, GIBCO Invitrogen Co) prior to treatment with trypsin-ethylenediaminetetraacetic acid (EDTA) (GIBCO Invitrogen Co) for 10 min. Culture medium with 10% FBS is used to neutralize the trypsin-EDTA solution. The cell suspension is centrifuged (1500 rpm, 10 min) and washed with culture medium. Cells are reseeded at an initial seeding density of 2×104 cells/cm2.

At different time points cells will be isolated and RNA extracted as described above. Using Real-time PCR mRNA levels of the above mentioned markers (COL2A1, COL1A1, Aggrecan, CRYAB and HSP27) will be evaluated at the different time points. Moreover, at the different time points cells will be grown in a pellet tissue culture system as described hereinafter, in attempt to restore the original phenotype. The ability to express a similar marker profile upon pellet tissue culture, compared to phenotypically stable SFBs, will be used to evaluate the phenotypical stability of the expanding cell culture.

### Pellet Tissue Culture System

SFBs are detached individually by trypsinization with 0.25% trypsin/0.2% EDTA. 0.3×106 cells are centrifuged at 500 g for 10 min in a 15 mL tube to form a pellet. The pellets are cultured in 24-well plates (2 mL medium per well) on a rotating shaker in a humid 5% CO2/95% air incubator with a defined medium, including High-Glucose DMEM, 40 mg/mL proline, 100 mM dexamethasone, 0.1 mM ascorbic acid 2-phosphate (Wako, Richmond, Va., USA), 100 U/mL penicillin, 100 mg/L streptomycin, and 1×ITS Premix [Collaborative Biomedical Products: insulin (6.25 mg/mL), transferrin (6.25 mg/mL), selenious acid (6.25 mg/mL), and linoleic acid (5.35 mg/mL), with bovine serum albumin (1.25 mg/mL)] with the supplementation of 10 ng/mL TGF-b1. The medium is changed every other day.

### REFERENCES

[1] Felson, D. T., Lawrence, R. C., Dieppe, P. A., Hirsch, R., et al., Ann Intern Med 2000, 133, 635-646.
[2] Dieppe, P. A. and Lohmander, L. S., Lancet 2005, 365, 965-973.
[3] Goldring, M. B., in: Klippel, J. H. (Ed.), Primer on the rheumatic diseases, Arthritis Foundation, Atlanta 2000, pp. 5-31.
[4] Haslbeck, M., Franzmann, T., Weinfurtner, D. and Buchner, J., Nat Struct Mol Biol 2005, 12, 842-846.
[5] Vicart, P., Caron, A., Guicheney, P., Li, Z., et al., Nat Genet. 1998, 20, 92-95.
[6] Berry, V., Francis, P., Reddy, M. A., Collyer, D., et al., Am J Hum Genet. 2001, 69, 1141-1145.
[7] Sun, Y. and MacRae, T. H., Febs J 2005, 272, 2613-2627.
[8] Lambrecht, S., Verbruggen, G., Verdonk, P., Elewaut, D. and Deforce, D., Osteoarthritis Cartilage, doi: 10.1016/j.joca.2007.06.005.
[9] Verbruggen, G., Wang, J., Wang, L., Elewaut, D. and Veys, E. M., Analysis of chondrocyte functional markers and pericellular matrix components by flow cytometry, Humana Press, Totowa, N.J. 2004, pp. 183-208.
[10] Guo, J. F., Jourdian, G. W. and MacCallum, D. K., Connect Tissue Res 1989, 19, 277-297.
[11] Verbruggen, G., Veys, E. M., Wieme, N., Malfait, A. M., et al., Clin Exp Rheumatol 1990, 8, 371-378.
[12] Livak, K. J. and Schmittgen, T. D., Methods 2001, 25, 402-408.
[13] Vandesompele, J., De Preter, K., Pattyn, F., Poppe, B., et al., Genome Biol 2002, 3, 34.
[14] Molloy, M. P., Herbert, B. R., Walsh, B. J., Tyler, M. I., et al., Electrophoresis 1998, 19, 837-844.
[15] Haslbeck, M., Franzmann, T., Weinfurtner, D. and Buchner, J., Nat Struct Mol Biol 2005, 12, 842-846.
[16] Kappe, G., Franck, E., Verschuure, P., Boelens, W. C., et al., Cell Stress Chaperones 2003, 8, 53-61.
[17] Arrigo, A. P., Simon, S., Gibed, B., Kretz-Remy, C., et al., FEBS Lett 2007.
[18] Xu, L., Chen, S, and Bergan, R. C., Oncogene 2006, 25, 2987-2998.
[19] Grimaud, E., Heymann, D. and Redini, F., Cytokine Growth Factor Rev 2002, 13, 241-257.
[20] Favet, N., Duverger, O., Loones, M. T., Poliard, A., et al., Cell Death Differ 2001, 8, 603-613.

1. A method to monitor the phenotypic stability of a cell culture of isolated pluripotent cells and/or chondrocytes, said method comprising monitoring the transient decrease in CRYAB expression, and wherein up to the increase of CRYAB expression, said cell culture is identified as a phenotypic stable cell culture.

2. The method according to claim 1, wherein said transient decrease in CRYAB expression is at least 30% of the initial CRYAB expression in said cell culture of isolated pluripotent cells and/or chondrocytes.

3. The method according to claim 1 further comprising monitoring HSP27 expression in said cell culture, wherein an increase in HSP27 expression to a level of at least 140% of the initial HSP27 expression in said cell culture is an indication of the dedifferentiation of said cell culture of isolated pluripotent cells and/or chondrocytes.

4. The method according to claim 3 wherein
a decrease in CRYAB expression down to about 60% to about 40% of the initial CRYAB expression and
an increase in HSP27 expression up to about 150% to about 250% of the initial HSP27 expression
identifies said cell culture of isolated pluripotent cells and/or chondrocytes as a phenotypic stable cell culture.

5. The method according to claim 1 wherein the phenotypic stability of the isolated chondrocytes is further characterised in their capability to retain COL2A1 and aggrecan expression upon reconstitution in their endogenous environment.

6. The method according to claim 5, wherein the capability of said cells to express COL2A1 and/or aggrecan is tested by expansion of said cells on a suitable carrier selected from the group consisting of carboxymethyl cellulose and other cellulose derivatives, alginates, gelatin, and polyvinyl pyrolidone.

7. The method according to claim 1 wherein the cell culture is a cell culture of isolated chondrocytes derived from skeletal tissue.

8. Use of a method according to claim 1 to identify cell culture conditions or treatments that enhance the phenotypic stability of a cell culture of isolated pluripotent cells and/or chondrocytes.

9. Use of CRYAB, HSP27 or of CRYAB and HSP27, in determining the chondrogenic capacity of a cell culture of isolated chondrocytes.

10. A method to identify compounds or cell culture conditions capable of enhancing the phenotypic stability of a cell culture of isolated pluripotent cells and/or chondrocytes, said method comprising applying the methods according to claim 1 in the presence and absence of the compound to be tested; and determining whether said compound is capable to prevent and/or delay the transient change in CRYAB expression.

11. A method to identify compounds capable of modulating the sensitivity of a cell culture of isolated pluripotent cells and/or chondrocytes for pro-inflammatory cytokines, said method comprising;
pre-treating said cells with one or more pro-inflammatory cytokines;
determining the expression levels of CRYAB and HSP27 at regular time intervals in a cell culture of said isolated and pretreated pluripotent cells and/or chondrocytes, both in the presence and absence of the compound or cell culture condition to be tested; wherein a compound or cell culture condition capable of preventing an increase in HSP27 expression and to delay or prevent the transient decrease in CRYAB expression is identified as a compound or cell culture condition that attenuates the sensitivity of isolated pluripotent cells and/or chondrocytes for pro-inflammatory cytokines.

12. A method according to claim 11 wherein the cells are treated with a pro-inflammatory cytokine selected from the group consisting of IL-1, IL-17, IL-18, IL-6, IL-8 or TNF.

13. A method according to claim 1 wherein the cell culture is a cell culture of isolated chondrocytes derived from skeletal tissue.

14. Use of a method according to claim 1 to obtain a phenotypic stable cell culture.

15. Use of a method according to claim 1 to obtain a cell culture with high chondrogenic capacity.

16. A phenotypic stable cell culture obtainable by claim 1.

17. A pharmaceutical composition comprising a cell culture of claim 16.

18. A cell culture according to claim 16 for use as a medicine.

19. Use of a cell culture according to claim 16 in the repair of connective tissue.

20. A pharmaceutical composition according to claim 17 for use as a medicine.
